SEQUENCE LISTING (14 sequences)

SEQ ID NO: 1 - DNA, 22 bp, Artificial Sequence
  Forward PCR primer for mir-34b/c
ggtygagtgattgtggyggggg

SEQ ID NO: 2 - DNA, 26 bp, Artificial Sequence
  Reverse PCR primer for mir-34b/c
cctccatcttctaaacrtctccctta

SEQ ID NO: 3 - DNA, 18 bp, Artificial Sequence
  Pyrosequencing primer for mir-34b/c
taatygtttttggaattt

SEQ ID NO: 4 - DNA, 25 bp, Artificial Sequence
  Forward PCR primer for SFRP1
gttttgttttttaaggggtgttgag

SEQ ID NO: 5 - DNA, 26 bp, Artificial Sequence
  Reverse PCR primer for SFRP1
ctccraaaactacaaaactaaaatac

SEQ ID NO: 6 - DNA, 19 bp, Artificial Sequence
  Pyrosequencing primer for SFRP1
gygtttggttttagtaaat

SEQ ID NO: 7 - DNA, 15 bp, Artificial Sequence
  Pyrosequencing primer for SFRP1
tygggagttgattgg

SEQ ID NO: 8 - DNA, 26 bp, Artificial Sequence
  Forward PCR primer for SFRP2
aatttyggattggggtaaaataagtt

SEQ ID NO: 9 - DNA, 27 bp, Artificial Sequence
  Reverse PCR primer for SFRP2
ttaaacaacaaacaaaaaaacctaacc

SEQ ID NO: 10 - DNA, 19 bp, Artificial Sequence
  Pyrosequencing primer for SFRP2
ygttttygttagtatttgg

SEQ ID NO: 11 - DNA, 25 bp, Artificial Sequence
  Forward PCR primer for DKK2
aagtaaagaggattggggagagagt

SEQ ID NO: 12 - DNA, 26 bp, Artificial Sequence
  Reverse PCR primer for DKK2
ttatcccctaactcacaaaaaacaac

SEQ ID NO: 13 - DNA, 20 bp, Artificial Sequence
  Pyrosequencing primer for DKK2
gagagagtagagagagagaa

SEQ ID NO: 14 - DNA, 20 bp, Artificial Sequence
  Pyrosequencing primer for DKK2
taagttygttttttaggtat

(19) United States
(12) Patent Application Publication
(10) Pub. No.: US 2012/0264120 A1
(43) Pub. Date: Oct. 18, 2012

(54) SPECIMEN FOR DETECTING INFILTRATIVE LARGE INTESTINE TUMORS

(75) Inventors: MINORU TOYOTA, SAPPORO-SHI (JP); Mutsumi Toyota, Sapporo-shi (JP); EIICHIRO YAMAMOTO, SAPPORO-SHI (JP); SEIKO KAMIMAE, SAPPORO (JP); SUZUKI HIROMU, SAPPORO-SHI (JP); HIROO YAMANO, AKITA-SHI (JP)

(73) Assignee: SAPPORO MEDICAL UNIVERSITY, SAPPORO-SHI (JP)

(21) Appl. No.: 13/407,645

(22) Filed: Feb. 28, 2012

Related U.S. Application Data
(63) Continuation-in-part of application No. PCT/JP2010/064715, filed on Aug. 30, 2010.
(60) Provisional application No. 61/238,106, filed on Aug. 28, 2009.

(51) Int. Cl. C12Q 1/68; G01N 33/48

(52) U.S. Cl. 435/6.11; 435/40.51; 435/6.14

(57) ABSTRACT

An object of the present invention is to provide a method for non-invasively diagnosing invasiveness or degree of invasion of colorectal tumors.

The present invention is characterized in that it enables to obtain a specimen that can be used to detect invasive colorectal tumors by spraying a washing fluid onto the colonic mucous layer of a subject to detach the mucus from the mucous layer, and collecting the detached mucus together with the washing fluid.

### TECHNICAL FIELD

This application is a continuation-in-part of, and under 35 U.S.C. §120 claims the benefit of priority to, international application Ser. No. PCT/JP2010/064715, filed on Aug. 30, 2010, which claims the benefit of priority to US provisional application Ser. No. 61/238,106, filed Aug. 28, 2009, the entire contents of both of which are hereby incorporated herein by reference.

The present invention relates to a specimen for diagnosing invasive colorectal tumors which is obtained by non-invasively detaching the colonic mucous layer, a kit for non-invasively detaching the colonic mucous layer, a method for collecting a specimen for diagnosing invasive colorectal tumors non-invasively, a method for detecting invasive colorectal tumors by non-invasively detaching the colonic mucous layer, a method for evaluating therapeutic effects of a drug and/or a therapeutic method by non-invasively detaching the colonic mucous layer, and a method for diagnosing the degree of invasion of invasive colorectal tumors. More specifically, the present invention relates to said specimen and kit for diagnosing invasive colorectal tumors, said method for detecting invasive colorectal tumors, and said method for evaluating therapeutic effects, wherein methylated DNA is used as a molecular marker.

### BACKGROUND ART

In Europe and the United States, colorectal cancer ranks high in cancer mortality. According to the prediction by statistical data in the United States in 2006, colorectal cancer is the third most commonly diagnosed cancer in both male and female, and its incidence increased annually by 1.8% from 1998 to 2002. In recent years, the number of patients with colorectal cancer is rapidly increasing in Japan as well. This is considered to be because Japanese diet has changed to European and American diet where meat is the center. In Japan, it is reported that approximately 100,000 people are annually diagnosed to have colorectal cancer, and approximately 41,000 people die. With respect to the number of death by organs, the number of colorectal cancer is the third largest following gastric cancer and lung cancer, and a further increase is being predicted. In particular, in females, colorectal cancer ranks the first among all malignant tumors, both in the number of patients diagnosed and the number of death. In males, colorectal cancer is predicted to be the third following lung cancer and liver cancer.

Epidemiologically, colorectal cancer is presumed to be caused by diet, in particular by excessive intake of animal fat and protein, rather than genetic predisposition; regarding the site in the large intestine, colorectal cancer easily develops in the sigmoid colon and the rectum.

However, different from other cancers, it is known that colorectal cancer is nearly 100% curable by surgery if detected early. Accordingly, colorectal cancer has been a subject of early cancer screening, and a number of test methods have been developed.

In addition, endoscopic surgeries such as endoscopic demucosation and endoscopic submucosal dissection are very effective for early cancer. Meanwhile, in invasive tumors, therapeutic methods such as laparotomy in combination with chemotherapeutic agents or radiation therapy, etc., are generally carried out. Therefore, development of a diagnostic method that can non-invasively evaluate invasiveness or invasion depth of cancer or tumor has been desired.

The large intestine has a five-layered structure consisting of, from the lumen side, mucosa, submucosa, muscularispropria, subserosa, and serosa; in the lower rectum, a three-layered structure excluding the serosa and subserosa is formed. Colorectal tumors originate from the mucosa, and infiltrate into deep layers as the tumors progress. Tumors in which their invasion is limited to the submucosa are called early cancer.

General test methods for digestive tract cancers, in particular colorectal cancer and rectum cancer, etc. include (i) fecal occult blood test, (ii) digital rectal examination, (iii) blood test, (iv) enema examination, (v) positron emission computed tomography (PET), (vi) endoscopy, (vii) capsule endoscopy, (viii) gene diagnosis using feces or biopsy sample.

However, these test methods aim at detection of early cancer, measurement of therapeutic effects, provision of materials for determining recurrence and metastasis, or definite diagnosis, and any of these methods cannot provide an index to determine the degree of invasion of cancer or tumor.

Fecal occult blood test is a method to indirectly predict occurrence of colorectal tumors by checking the presence/absence and the amount, etc. of the blood in feces, utilizing a peroxidase activity in human hemoglobin or a monoclonal antibody against human hemoglobin, and this is a simple, inexpensive and non-invasive test method. However, effectiveness of occult blood test is decreased by the fact that bleeding from a colorectal tumor is intermittent, which increases false-negative rates. For example, approximately 50% of the patients diagnosed to have a colorectal tumor are tested negative for fecal occult blood. In addition, since the amount of bleeding from small colorectal tumors with a diameter of less than 20 mm is as small as 1-2 ml per day, blood is not always detected by occult blood test. Furthermore, because positive results can be made by a number of causes other than colorectal tumors, including gingivitis, hemorrhoids, ulcers, and intestinal bleeding due to aspirin use, only 3-5% of the subjects tested positive for fecal occult blood actually have colorectal tumors, and many false-positive subjects are included in the subjects tested positive for fecal occult blood. Thus, fecal occult blood test is not a specific screening test for tumors such as colorectal tumors, and it is not necessarily sufficient as a preliminary diagnostic method of colorectal tumors. Moreover, it is impossible to determine the degree of invasion of colorectal tumors by fecal occult blood test.

By means of digital rectal examination, it is possible to detect tumors located in the far end of the rectum/colon by digital examination, but not tumors located in interior regions. Furthermore, diagnosis of the degree of invasion of colorectal tumors by digital rectal examination is not possible.

Blood tests are diagnostic method of diagnosing colorectal tumors by measuring tumor markers in the blood sample of a subject and thereby determining the amount or concentration of the tumor markers.

There are two types of tumor markers: a tumor marker, the detection of which means diagnosis of colorectal cancer, such as fetal proteins (AFP, CEA, etc.), carbohydrate antigens (CA19-9, serial Tn, etc.), and ectopically-produced substances (hormones and tumor isozyme, etc.), and a tumor marker, the detection of its genetic mutation and genetic recombination provides information, such as oncogenes (ras, erbB, etc.), tumor suppressor genes (p53, etc.), and gene rearrangement (BCR-ABL, etc.).

However, tumor markers in the blood have the following drawbacks: they may be positive even in the absence of colorectal cancer, they may not be positive until colorectal cancer grows to a certain extent, and they may not be positive even for advanced colorectal cancer. Therefore, tumor markers of colorectal cancer do not show effects that lead to early detection or definite diagnosis of cancer; at present, they are used as auxiliary diagnosis and used for measurement of therapeutic effects, and as one of the criteria for detection of recurrence and metastasis. In addition, degrees of invasion of colorectal tumors cannot be determined by concentrations of tumor markers in the blood.

Enema examination is a method to examine intestinal surface irregularity by X-ray, by injecting barium into the large intestine to adhere it onto the intestinal mucosal surface. However, enema examination has problems that it is costly and subjects must bear a large burden, with a risk of complications. For example, in the enema examination, after a subject takes edema diet comprising low fat and low residue, the subject undergoes a pretreatment to eliminate the contents of the large intestine by administration of a laxative (saline purgative and contact laxative). Moreover, since enema examination only checks irregular morphology of the intestinal lumen, the degree of invasion of colorectal tumors cannot be determined by the enema examination.

Positron emission computed tomography (PET) is a method wherein a drug labeled with a positron-emitting radionuclide is administered to a subject and the quantities of the drug consumed at various sites of the body are investigated. For example, fluorodeoxyglucose labeled with 18F which is a kind of sugar and has a characteristic of accumulating in tumors is administered to a subject as a PET agent, then gamma rays are observed from external of the body, and distribution of the labeled substance inside the body is imaged to investigate its kinetics in the body, thereby determining the location and size of lesions.

PET usually requires a cyclotron and needs an expensive equipment that costs more than 1,000,000,000 yen. In addition, radiation exposure cannot be avoided upon execution of PET. Moreover, although approximate size of a tumor can be measured by PET, the degree of invasion of a colorectal tumor cannot be determined.

Endoscopy is a method to investigate inside of the large intestine directly by an endoscope. Endoscopy has high sensitivity and specificity in the detection of colorectal tumors. In addition, endoscopy is advantageous in that it can excise early cancers and precancerous polyps. Furthermore, endoscopy is advantageous in that it can collect tissues for diagnosis by biopsy (tissue biopsy). However, in endoscopy, since surface of the intestine is observed from the lumen side, it is impossible to determine the degree of invasion of colorectal cancer. In fact, among tumors detected by colonoscopy, a large number of tumors that have a small lesion but invade into the submucosal tissue of the large intestine are included.

In tissue biopsy by endoscopy, only the tissue at a “point” is evaluated, and there is a limitation in extending the points to an “area.” Endoscopic tissue biopsy evaluates only a part of the lesion, and therefore, definite diagnosis of colorectal cancer is impossible depending on the biopsy site, or depending on the removal site of specimens even for lesions of endoscopic mucosal ablation. Moreover, it is impossible to accurately determine the invasion depth of tumors using endoscopic tissue biopsy.

In general, a definite diagnosis of colorectal cancer is made by histopathological diagnosis using biopsy materials, so that preparation of histopathological specimens based on detailed endoscopic observation is required for definite diagnosis of colorectal tumors. However, preparation of such histopathological specimens requires a great deal of expertise, and therefore not all physicians performing endoscopy can prepare such specimens.

Regarding minute tumors and tumors before colorectal mucosectomy, biopsy makes their pathological profiles after surgery unstable, and hence, whether to perform endoscopic ablation or open abdominal surgery has been determined based only on the endoscopic observation of size and shape as well as pit pattern diagnosis using magnifying endoscopy.

Capsule endoscopy is an examination method of gastrointestinal tracts, wherein, a patient swallows a capsule containing a tiny camera which automatically takes pictures while it passes through the gastrointestinal tract, and the image information taken is wirelessly transmitted to outside of the body. Capsule endoscopy has the following drawbacks: the resolution of images is lower than that of general endoscopy, and since it is an automatic shooting, sufficient observation inside the folds that is the target of fine examination is impossible, and biopsy and polypectomy are impossible. Accordingly, at present capsule endoscopy is mainly used for examination of the small intestine, the observation of which is difficult by a general endoscope. Furthermore, since the intestinal surface is observed from the lumen side by capsule endoscopy as well, it is impossible to determine the degree of invasion of colorectal tumors by capsule endoscopy.

Gene diagnosis using feces or biopsy sample is a method of diagnosis of colorectal cancer by examining genes of tumor cells detached in the feces or tumor cells contained in the biopsy sample. Genetic mutation and hypermethylation of DNA occurred in tumor cells are stable information because once occurred, it is difficult to return to normal state. Therefore, when specimens are appropriate, gene diagnosis is a highly accurate diagnostic method.

However, when gene diagnosis is performed using feces, because various bacteria and nucleic acids derived from normal cells are present in the feces, the relative amount of genes derived from tumor cells collected from the feces becomes very small (approximately 0.05%), resulting in a problem that accurate diagnosis is difficult. For example, in Non-patent Literatures 1 and 2, it is described that a certain outcome has been obtained by a method of diagnosing colorectal tumors using DNA in the feces. However, these methods have problems in the possibility of diagnosis and diagnostic accuracy, and they are still far from practical application.

In contrast, when gene diagnosis is performed using biopsy samples, results vary widely depending on the collection site, which is problematic. In addition, regarding minute tumors and tumors before colorectal mucosectomy, biopsy causes fibrotic response and thermocoagulation denaturation, leading to a problem of variable pathological profiles after surgery.

Changes in genes that are the subject of gene diagnosis of colorectal tumors include mutation of oncogenes such as ras, erbB, etc., mutation of tumor suppressor genes such as p53, etc., and detection of gene rearrangement such as BCR-ABL, etc., as well as epigenetic modification such as hypermethylation in the promoter CpG island regions of tumor suppressor genes.

When a CpG island present in the promoter region of a tumor suppressor gene is methylated, transcription of this tumor suppressor gene is inactivated, leading to ineffectiveness in the control of cell growth and causing progression of cell proliferative diseases such as cancer. For example, in cancer cells, expression of the following genomic genes is inhibited by hypermethylation in the promoter CpG island regions thereof: SFRP1, SFRP2, DKK2, hsa-mir-34b/c, p16INK4A, E-cadherin, hMLH1,14-3-3 sigma, BNIP3 that is one of BH3 Only family gene, ubiquitin ligase CHFR, CITTA that is a transcriptional coupling factor of MHC class II molecules, IGFBP7 that is a negative regulatory gene of BRAF in colorectal cancer, Histone H3K27, HRK that is an apoptosis-related gene, CACNA1G, COX2, DFNA5, and RASSF2 that is a regulatory gene of Ras; and it is reported that they are useful in diagnosis of colorectal tumors.

Hypermethylation can be detected from a minute amount of DNA, and it is stable information in that once hypermethylation occurs, it hardly returns to a normal state naturally, so that hypermethylation is considered to be useful as an index for gene diagnosis. However, while it is possible to determine the presence of tumors by gene diagnosis using feces or biopsy sample, determination of the degree of invasion of tumors is impossible.

In recent years, with advancement of endoscopic technology, the number of cases wherein colorectal tumors are cured by endoscopy without surgery has been increasing. However, there are problems such as when biopsy is performed in advance, endoscopic treatment becomes difficult, or accurate pathological diagnosis of ablation specimens becomes difficult. In some endoscopy-specialized facilities, accurate diagnosis is performed by extremely detailed magnifying endoscopic observation without biopsy, and by microscopic observation of ablation specimens; however, at present this can be performed only at limited facilities.

Thus, to date there has been no method which accurately determines genetic features of colorectal cancer cells without biopsy. In particular, there is no known method for analyzing DNA methylation of colorectal tumor cells.

### CITATION LIST

### Non-Patent Literature

Non-patent Literature 1: Ahlquist DA et al., Gastroenterology. 2000 Nov; 119 (5):1219-27

Non-patent Literature 2: Osborn N K et al., Gastroenterology. 2005 Jan; 128 (1):192-206

### SUMMARY OF INVENTION

### Technical Problem

Therefore, an object of the present invention is to provide a specimen for diagnosing invasive colorectal tumors obtained by non-invasively detaching the colonic mucous layer, a kit for non-invasively detaching the colonic mucous layer, a method for collecting a specimen for diagnosing invasive colorectal tumors by non-invasively detaching the colonic mucous layer, a method for detecting invasive colorectal tumors by non-invasively detaching the colonic mucous layer, a method for evaluating therapeutic effects of a drug and/or a therapeutic method by non-invasively detaching the colonic mucous layer, and a method for diagnosing the degree of invasion of invasive colorectal tumors, which do not have the above-mentioned drawbacks.

### Solution to Problem

The present inventors have devoted themselves to the research to solve the above problems, and surprisingly, they have found that it is possible to obtain a specimen for diagnosing invasive colorectal tumors by spraying a washing fluid onto the colonic mucous layer of a subject to detach the mucus from said mucous layer, and collecting the detached mucus together with the washing fluid; and thus, the inventors have accomplished the present invention.

Namely, the present invention relates to the following specimen, kit, or method.

[1] A colonic-mucous-layer detachment fluid comprising the colonic mucus detached from the colonic mucous layer, which does not substantially comprise a content existing in the digestive tract from the oral cavity to the small intestine and a component derived from said digestive tract.
[2] The colonic-mucous-layer detachment fluid according to
[1] for diagnosing invasive colorectal tumors, obtained by spraying a washing fluid directly onto the colonic mucous layer to detach the colonic mucus, and collecting the colonic mucus detached from the directly-sprayed site together with the washing fluid.
[3] A method for collecting a colonic-mucous-layer detachment fluid for diagnosing invasive colorectal tumors, comprising:
(a) spraying a washing fluid directly onto the colonic mucous layer, and
(b) collecting the colonic mucus detached from the directly-sprayed site together with the washing fluid.
[4] The method according to [3], wherein the washing fluid is a physiological isotonic solution.
[5] The method according to [4], wherein the physiological isotonic solution is saline.
[6] The method according to any one of [3] to [5], wherein the spraying is carried out at a flow rate of 2-10 ml/s.
[7] The method according to any one of [3] to [5], wherein the washing fluid is sprayed without pre-washing.
[8] A method for detecting an invasive colorectal tumor, comprising:
(a) determining one or more disease-related marker levels in the colonic-mucous-layer detachment fluid according to [1] or [2], and
(b) determining the presence or absence of an invasive colorectal tumor based on the disease-related marker levels.
[9] The method according to [8], wherein the colonic mucous layer is a colonic mucous layer comprising a site of lesion.
[10] The method according to [9], wherein the site of lesion is a site of lesion suspected of tumor invasion.
[11] The method according to any one of [8] to [10], wherein the determination of a disease-related marker level is carried out by cytological diagnosis.
[12] The method according to any one of [8] to [10], wherein the disease-related marker is methylated DNA.
[13] The method according to [12], wherein the methylated DNA is a methylated DNA in the promoter region of one or more genes selected from the group consisting of SFRP1, SFRP2, DKK2, and hsa-mir-34b/c.
[14] The method according to any one of [8] to [10], wherein the disease-related marker is K-RAS mutation.
[15] The method according to any one of [8] to [14], comprising determining two or more disease-related marker levels.
[16] A method for diagnosing the degree of invasion of an invasive colorectal tumor, comprising:
(a) determining one or more disease-related marker levels in the colonic-mucous-layer detachment fluid according to [1] or [2], and
(b) determining the degree of invasion based on the disease-related marker levels.
[17] The method according to [16], wherein the disease-related marker level is a DNA methylation level.
[18] A method for evaluating therapeutic effects of a drug and/or a therapeutic method, comprising:
(a) determining one or more disease-related marker levels A in the colonic-mucous-layer detachment fluid according to [1] or [2] before treatment by a drug and/or a therapeutic method,
(b) after the treatment by the drug and/or the therapeutic method, determining one or more disease-related marker levels B in the colonic-mucous-layer detachment fluid according to [1] or [2] which correspond to said A, and

(c) comparing said A with B.

[19] A kit for collecting the colonic-mucous-layer detachment fluid according to [2], containing at least one sealable specimen collection container to collect the washing fluid, and a preservative solution.
[20] The kit according to[19], further containing a tool for treating the collected colonic-mucous-layer detachment fluid.
[21] The kit according to [20] , wherein the tool for treating the colonic-mucous-layer detachment fluid is one or more primers used for the detection of methylated DNA.

### Effects of Invention

The specimen, kit and method of the present invention use a colonic-mucous-layer detachment fluid obtained by washing the colonic mucous layer that adheres to the mucosa of the large intestine, in particular the mucosa of the lesion of a subject, and therefore the present invention is fundamentally different from conventional methods using biopsy samples of the colonic mucosa.

Surprisingly, the inventors have found that a larger amount of tumor cells can be collected from the colonic-mucous-layer detachment fluid, as the degree of invasion of cancer or tumor increases. Therefore, according to the specimen, kit, and method of the present invention, as the degree of invasion of cancer or tumor increases, a larger amount of tumor cells can be collected from the colonic mucous layer, so that more accurate diagnosis of degree of invasion can be made by cytological diagnosis using the tumor cells and by examination of disease-related markers including DNA testing. Accordingly, the specimen, kit, and method of the present invention are useful as a specimen, kit and method for diagnosing the degree of invasion of cancers or tumors, which can provide various beneficial effects that had been impossible to achieve by conventional methods.

Namely, because the specimen, kit, and method of the present invention can be performed by simple equipment, and a washing fluid that had been disposed of to date is used as a material, various risks that may be caused by other test methods (for example, risk of bleeding (biopsy), allergy, extended test time (magnifying endoscopy), expensive equipment(magnifying endoscopy, NBI) and others) do not occur, and since the collection of a specimen is easy, almost no burden is placed on physicians performing endoscopy and clinical nurses assisting them; furthermore, a commercially available endoscopic apparatus can be used as is, eliminating the necessity of new equipment investment.

In addition, in the biopsy wherein a part of a tumor is collected and examined, evaluation of only a “point” where the specimen has been collected is possible; whereas with the specimen, kit and method of the present invention, it becomes possible to evaluate the lesion as an “area” by washing its entire mucosal surface. This would be beneficial in the following case: for example, a tumor tissue on the whole is not homogenous, and a tissue collected by biopsy does not necessarily reflect the characteristic of the entire tumor; accordingly, when a specimen is collected from a tumor having a characteristic of invasive tumor, this specimen may happen to strongly exhibit the characteristic of noninvasive tumor. Even in such a case, since the present invention enables evaluation of an “area,” i.e., the entire tumor, more accurate evaluation is possible. Furthermore, since the tissue is not injured as in the case of biopsy, tests can be performed for subjects whose hemostatic function and wound healing ability decrease or for subjects who are orally taking drugs affecting blood stanching and wound healing, such as antiplatelet drugs and anticoagulants. Since many of the subjects requiring endoscopy are relatively elderly persons, the ratio of those who taking such drugs is high; thus, such an advantage is extremely important.

According to the specimen, kit and method of the present invention, it is possible to perform disease-related marker tests including cytological diagnosis and DNA diagnosis without injuring tumors prior to surgery. Accordingly, using the specimen, kit and method of the present invention, accuracy of estimating characteristics and degree of invasion of tumors can be significantly improved. Furthermore, using the specimen, kit and method of the present invention, it becomes possible to diagnose genetic profiles of colorectal tumors before surgery.

With the specimen, kit and method of the present invention, it is possible to detect genetic or epigenetic abnormalities of genomic DNA. Furthermore, sensitivity of various drugs including anticancer agents can be examined using specimens collected by the method of the present invention. Moreover, using the specimen and method of the present invention, evaluation of therapeutic effects of drugs and/or therapeutic methods becomes possible. With the specimen and method of the present invention, it becomes possible to predict recurrence.

According to the analysis of disease-related markers including hypermethylation using a colonic-mucous-layer detachment fluid using the specimen, kit and method of the present invention, accurate diagnosis of invasive colorectal tumors by a simple and non-invasive method becomes possible at any facilities. Thus, the specimen, kit and method of the present invention are extremely useful.

### BRIEF DESCRIPTION OF DRAWINGS

FIG. 1 shows photographs showing the process of spraying a washing fluid onto the colonic mucous layer and thereby detaching the mucus from the mucous layer. “A” shows a tumor part before spraying the washing fluid, “B” shows spraying the washing fluid onto the tumor part, and “C” shows the tumor part after spraying the washing fluid.

FIG. 2 shows photographs of cells obtained by spraying a washing fluid onto the colonic mucous layer and thereby detaching the mucus from the mucous layer. “Method 1” shows a photograph of cells obtained by spraying water onto the colonic mucous layer and detaching the mucus from the mucous layer. “Method 2” shows a photograph of cells obtained by spraying saline onto the colonic mucous layer and detaching the mucus from the mucous layer. The cells were stained with hematoxylin-eosin.

FIG. 3 shows diagrams of percentage of methylated DNA in the large intestine of patients with invasive tumor, patients with non-invasive tumor, and noncancer patients in the training set, compared between biopsy sample and colonic-mucous-layer detachment fluid. In the diagrams, each point shows each case. The vertical axis indicates the percentage of methylated DNA in the promoter CpG island regions of each gene miR-34b/c, SFRP1, SFRP2, and DKK2. “Biopsy samples” indicates biopsy sample, “Washing fluids” indicates colonic-mucous-layer detachment fluid. “IC” indicates invasive tumor, “NI” indicates non-invasive tumor, “Normal” indicates noncancer patient. Numerical values after “N=” indicate the number of cases. “NS” means that there is no significant difference, and numerical values after “P<” indicate the level of significance. For example, “P<0.0001” means that there is a statistically significant difference with a significance level of 0.01%. Here, horizontal lines in the diagrams show mean values.

FIG. 4 is a diagram showing receiver operating characteristic curves (ROC curves) of the training set. By drawing ROC curves and determining best cut-off values, the most appropriate threshold value for distinguishing between invasive tumor and non-invasive tumor, using the percentage of methylated DNA of each gene in a specimen (Method 2) collected by spraying saline as a washing fluid, can be determined. mir-34b/c, SFRP1, SFRP2 and DKK2 indicate the gene name. The vertical axis “sensitivity” indicates “sensitivity”, and the horizontal axis “1-specificity” indicates “1-specificity”. “AUC” indicates the area under the ROC curve. “Best cut-off” indicates the most appropriate threshold value, i.e., “best cut-off” to distinguish between invasive tumor and non-invasive tumor. In the figure, numerical values listed to the right of “sensitivity” indicate sensitivities at the “best cut-off,” and numerical values listed to the right of “specificity” indicate specificities at the “best cut-off.”

FIG. 5 shows ROC curves of percentage of methylated DNA when tumors were classified by their size: diameter of less than 20 mm (left diagram) and diameter of 20 mm or more (right diagram). In the left diagram, for the case of tumors with a diameter of less than 20 mm, ROC curves of the percentage of methylated DNA of each gene mir-34b/c and SFRP1 are shown; in the right diagram, for the case of tumors with a diameter of 20 mm or larger, ROC curves of the percentage of methylated DNA of each gene mir-34b/c and DKK2 are shown. “Sensitivity” of the vertical axis indicates “sensitivity,” and “1-specificity” of the horizontal axis indicates “1-specificity.”

FIG. 6 is one example showing the determination process of diagnosing presence/absence of invasive tumors, using the percentages of methylated DNA of multiple genes as indices. In the figure, “Tumor size 20 mm≦” indicates the determination criterion that the tumor diameter is 20 mm or more. “mir34b/c 15%<” indicates the determination criterion that the percentage of methylated DNA of mir34b/c gene is more than 15%. “SFRP1 51%<” indicates the determination criterion that the percentage of methylated DNA of SFRP1 gene is more than 51%. “DKK2 1 0%<” indicates the determination criterion that the percentage of methylated DNA of DKK2 gene is more than 10%. “SFRP2 10%<” indicates the determination criterion that the percentage of methylated DNA of SFRP2 gene is more than 10%. “Yes” existing in the center of bar lines means that the criterion of its left side is satisfied; similarly, “No” existing in the center of bar lines means that the criterion of its left side is not satisfied. “Invasive caner” indicates diagnostic result of invasive tumor. “Non-invasive tumor” indicates diagnostic result of non-invasive tumor. The denominator of a numerical value to the right of “Yes” in the box indicates the number of cases that satisfy the criterion in the box, and the numerator indicates the number of cases of invasive tumors among the cases satisfying the criterion in the box. The denominator of a numerical value to the right of “No” in the box indicates the number of cases that do not satisfy the criterion in the box, and the numerator of the numerical value to the right of “No” in the box indicates the number of cases of invasive tumors among the cases that do not satisfy the criterion in the box. Numerical values after “P<” indicate the level of significance. For example, “P<0.001” means that there is a statistically significant difference with a significance level of 0.1%.

FIG. 7 shows diagrams for investigating the correlation between the percentage of methylated DNA of each gene in a specimen collected by spraying saline as a washing fluid (Method 2) and the percentage of methylated DNA of each gene in biopsy sample. In the figure, each point shows each case. “wash” on the vertical axis indicates the “percentage of methylated DNA in the colonic-mucous-layer detachment fluid obtained by spraying saline onto tumors,” and “biopsy” on the horizontal axis indicates the “percentage of methylated DNA in the biopsy sample.” Solid lines in the figure show regression lines by analysis of covariance, and dotted lines above and below the solid lines show confidence limits with a confidence level of 95%. Numerical values to the right of “R=” indicate Pearson's correlation coefficients, and values to the right of “P=” indicate risk rates.

FIG. 8 shows diagrams of the percentage of methylated DNA in the large intestine of patients with invasive tumor, patients with non-invasive tumor, and noncancer patients in the test set, in which data from biopsy samples and data from colonic-mucous-layer detachment fluid are compared. In the diagrams, each point represents each case. The vertical axis indicates the percentage of methylated DNA in the promoter CpG island regions of respective gene, miR-34b/c, SFRP1, SFRP2, and DKK2 . Terms and symbols in the figure have the same meanings as those described in FIG. 3.

FIG. 9 shows diagrams of ROC curves of the test set. The left figure represents the ROC curve of the entire samples. The middle figure represents the ROC curve for miR34b/c when tumor sizes are 25 mm or more, and the right figure represents the ROC curve for SFRP1 when tumor sizes are less than 25 mm.

FIG. 10 is one example of determination process of diagnosing presence/absence of invasive tumors, using the percentages of methylated DNA of multiple genes as indices. Terms and symbols in the figure have the same meanings as those described in FIG. 6.

### EMBODIMENTS FOR CARRYING OUT THE INVENTION

Hereinafter, preferred embodiments of the present invention are described in detail.

The present invention relates to a colonic-mucous-layer detachment fluid which does not substantially comprise a content existing in the digestive tract from the oral cavity to the small intestine and a component derived from said digestive tract, in particular, a specimen that is a colonic-mucous-layer detachment fluid for diagnosing invasive colorectal tumors which is obtained by non-invasively detaching the colonic mucous layer, a kit for non-invasively detaching the colonic mucous layer, a method for detecting invasive colorectal tumors by non-invasively detaching the colonic mucous layer, and a method for evaluating therapeutic effects of a drug and/or a therapeutic method by non-invasively detaching the colonic mucous layer.

In the present invention, “colonic mucous layer” refers to a layer of mucus present on the surface of the mucosa of the intestinal lumen.

In the present invention, “colonic-mucous-layer detachment fluid” refers to a liquid composition obtained by spraying a liquid onto the colonic mucous layer and detaching the mucus from said mucous layer, said fluid comprising the sprayed liquid and the detached mucus. The sprayed liquid typically is a washing fluid. Accordingly, the colonic-mucous-layer detachment fluid preferably comprises the colonic mucus and the washing fluid. Upon detachment of the mucus from the mucous layer, tissue cells at the site where the mucus was present may be simultaneously exfoliated.

Therefore, the colonic mucus may comprise such exfoliated cells. In the present invention, “components derived from the digestive tract” include such mucus and exfoliated cells.

As mentioned above, the present inventors have newly found that, in a colonic lesion, in particular at a tumor site, a large number of cells exfoliate simultaneously with the detachment of the mucus, and the number of exfoliated cells becomes larger as the degree of invasion of tumor increases. Then, they have found that it is possible to perform diagnosis of tumors using these exfoliated cells, including diagnosis using disease-related markers such as cytological diagnosis or DNA diagnosis, and accomplished the present invention.

The colonic-mucous-layer detachment fluid of the invention does not substantially comprise a content existing in the digestive tract from the oral cavity to the small intestine, i.e., the digestive tract other than the large intestine, and a component derived from the digestive tract other than the large intestine. Here, “does not substantially comprise” means that no such content and component are included, or such content and component are contained, if any, at a level that does not adversely affect the diagnosis of invasive colorectal tumors, or preferably, at a level that is impossible to detect.

As mentioned above, the colonic-mucous-layer detachment fluid of the invention comprise sex foliated tumor cells derived from colonic lesions; using these exfoliated cells, it is possible to perform diagnosis of tumors, including diagnosis using disease-related markers such as cytological diagnosis and DNA diagnosis. Therefore, the colonic-mucous-layer detachment fluid of the invention can be used as a specimen for diagnosing invasive colorectal tumors. In addition, the colonic-mucous-layer detachment fluid of the invention comprises exfoliated tumor cells at a level that enables diagnosis of invasive colorectal tumors. A “level that enables diagnosis of invasive colorectal tumors” can be expressed, for example, by an amount of DNA that can be collected from tumor cells, but it is not limited thereto. Accordingly, as an example of “level that enables diagnosis of invasive colorectal tumors,” it may refer to a specimen for diagnosing invasive colorectal tumors, comprising tumor cells from which at least 10 μg or more, preferably 12 μg or more, and more preferably 15 μg or more of DNA can be collected.

The colonic-mucous-layer detachment fluid of the invention does not substantially comprise a component or content derived from the digestive tract other than the large intestine. When a washing fluid spreads through the digestive tract using an orally administered intestinal-tract washing agent, etc., then generally, components or contents derived from the digestive tract from the oral cavity to the small intestine are mixed in the collected washing fluid; therefore, it is impossible to obtain the colonic-mucus-layer detachment fluid of the present invention using such a method. Therefore, preferably, a washing fluid is directly sprayed onto the colonic mucous layer to detach the colonic mucus, and the colonic mucus detached from the directly-sprayed site is collected together with the washing fluid. Thus, the method for collecting said colonic-mucous-layer detachment fluid is also encompassed by the present invention.

The washing fluid used in the present invention is preferably a liquid that can detach the colonic mucous layer without injuring the mucosa of the large intestine; from the viewpoint of small load on the colonic mucosa by the osmotic pressure, etc., it is more preferably an isotonic solution, and from the viewpoints of ease of preparation and low toxicity, furthermore preferably saline. In addition, the washing fluid used in the present invention may comprise any additives such as pigments, antibiotics, neutral buffer compositions, chelating agents and additives for preservation, as long as the washing fluid can detach the colonic mucous layer without injuring the mucosa of the large intestine.

In the present invention, the washing fluid is sprayed onto the colonic mucous layer at a rate that can detach the colonic mucous layer without injuring the mucosa of the large intestine. When the rate is too large, a possibility that the tumor site bleeds and contaminants mix into the specimen increases; when the rate is too low, the mucus does not detach well. Examples of such appropriate rate include, but are not limited to, 2 ml/s to 10 ml/s, for example 3 ml/s to 8 ml/s, for example 4 ml/s to 6 ml/s, etc. In the present invention, the large intestine may be pre-washed prior to spraying a washing fluid. However, from the viewpoint of ease of treatment, etc., a washing fluid is preferably sprayed without pre-washing. Furthermore, a washing fluid may be directly sprayed onto the site suspected of invasion of cancer or tumor to obtain a colonic-mucous-layer detachment fluid of the invention.

The washing fluid used in the present invention is preferably in an amount of 10-100 ml, more preferably 15-50 ml, furthermore preferably 17-30 ml, and most preferably 20 ml.

The method for collecting the washing fluid in the present invention is not particularly limited; when an endoscopic apparatus is used, for example, a method wherein at least one specimen collection container that can be sealed is connected to an operating part in an aspiration tube, connector, aspiration tank or aspirator, or the parts therebetween, may be included. When ease of washing and sterilization and risk of contamination are considered, the specimen collection container is preferably connected between the connector and the aspiration tank. In addition, from the viewpoints of operability and ease of washing, the specimen collection container is preferably connected in a detachable manner. Typically, for example, the aspiration tube that connects the connector and the aspiration tank is detached from the aspiration-tank side, and this tube is connected to the inflow-side connector of the specimen collection container, and the outflow-side connector of said container is connected to the aspiration tank. The endoscopic apparatus is preferably equipped with a member to support the specimen collection container. Only one specimen collection container mentioned above may be connected, or a plurality of containers may be connected in series or in parallel.

Moreover, the collected colonic-mucous-layer detachment fluid is, immediately after the collection, preferably subjected to the subsequent treatment for diagnosis, but the specimen can be stored using a preservative solution such as cell preservative solution and DNA preservative solution. Examples of such preservative solution may be those that can suppress degradation of nucleic acids, and preferably include SDS, EDTA or Tris, etc., which have effects to denature nucleolytic enzymes and effects to suppress action of nucleolytic enzymes by means of chleating specific ions. Suchpreservative solutions are commercially available, and they may also be used. As the commercially available preservative solutions, for example, Thinprep pap test from Cytyc corporation may be used.

Another aspect of the present invention relates to a method for detecting invasive colorectal tumors using the above colonic-mucous-layer detachment fluid. In one embodiment of such aspect, the present invention provides a method for detecting an invasive colorectal tumor, comprising: (a) determining one or more disease-related marker levels in the above colonic-mucous-layer detachment fluid, and (b) determining the presence or absence of an invasive colorectal tumor based on the disease-related marker levels.

In the present invention, “disease-related markers” encompass all that are characteristically exhibited in a disease; by detecting disease-related markers or by determining disease-related marker levels, the subject is determined to have the disease. The disease-related markers in the present invention mean that, although they differ depending on diseases, typically, disease-related markers appearing in invasive colorectal tumors. Examples of the “disease-related markers” of the present invention include not only tumor makers in a general sense, but also DNA and/or RNA specifically observed in cells of a subject having the disease, in particular, cells at a site of the disease, which include mutated DNA and modified DNA. The DNA specifically observed in tumor cells may include, but are not limited to, DNA and/or RNA related to genes that are specifically expressed in tumor cells such as oncogenes, mutated tumor-suppressor genes, as well as abnormally modified DNA such as abnormally methylated DNA. Furthermore, morphological characteristics such as dysplasia of blood vessels and atypical cells etc. are also included in the “disease-related markers” of the present invention.

The disease-related markers used in the present invention may be any disease-related markers as long as they can diagnose invasive colorectal tumors using a colonic-mucous-layer detachment fluid of invasive colorectal tumors. Examples of the disease-related markers used in the present invention include, but are not limited to: those that exhibit morphological characteristics of cells, for example grade of cellular atypism, obtained by histological diagnosis of the cells stained with, e.g., hematoxylin-eosin staining or Papanicolaou staining (cytological diagnosis); tumor makers, the detection of which can be used for the diagnosis of colorectal tumors, such as fetal proteins (AFP, CEA, etc.), carbohydrate antigens (CA19-9, serial Tn, etc.), ectopically-produced substances (hormones and tumor isozyme, etc.) and others; and oncogenes and tumor suppressor genes including cancer-related genes such as APC, K-RAS, H-RAS, N-RAS, erbB, p53, P16, BCR-ABL, CHFR, RASSF family, SFRP family, MINT Family, MGMT, RUNX family, SMAD family and PRDM family, EBV and its related genes, and CMV and its related genes, as well as their exprsesion products; and abnormally modified DNA such as methylated DNA present in the promoter region of a gene such as SFRP1, SFRP2, DKK2, hsa-mir-34b/c, p16INK4A, E-cadherin, hMLH1, 14-3-3 sigma, BNIP3, CHFR, CIITA, IGFBP7, Histone H3K27, HRK, CACNA1G, COX2, DFNA5, and RASSF2. Furthermore, detection of a mutation of an oncogene or tumor suppressor gene itself, such as the mutation of codons 12 and 13 in exson 2 of K-RAS gene, and detection of modified nucleotides such as the detection of methylcytosine in methylated DNA, are also encompassed in the detection of disease-related markers of the present invention.

In the present invention, “disease-related marker level” refers to, but is not limited to, histological evaluation scores such as grade of cellular atypism, concentrations of fetal proteins, carbohydrate antigens or ectopically-produced substances in a body fluid such as the blood, etc., amounts of mutated oncogenes or tumor suppressor genes contained in a specimen, levels of methylation in the promoter CpG island regions of tumor-related genes contained in a specimen, etc. “Determining a disease-related marker level” means determining the level, the amount of existence, and the ratio of existence or the concentration of such a disease-related marker by observing or measuring the disease-related maker present in a sample. For example, when the disease-related marker is a morphological characteristic, to find a morphology that is characteristic to the disease by observing the morphology is included in “detecting presence/absence of the disease-related marker,” and to classify the degree of abnormality of such morphology is included in “determining the disease-related marker level.” Furthermore, to detect presence/absence of the disease-related marker is also included in “determining the disease-related marker level.”

On the basis of the disease-related marker levels determined as described above, it is possible to determine presence or absence of an invasive colorectal tumor according to the method of the present invention. Criteria for determination vary depending on disease-related markers employed, and any judgment criteria known in the art may be adopted. Examples include, but are not limited to, that the presence of an invasive colorectal tumor is determined when mutated K-RAS is detected. In the present invention, “determining the presence of an invasive colorectal tumor” and “detecting the presence of an invasive colorectal tumor” are used interchangeably. In addition, since “detecting the presence of an invasive colorectal tumor” means essentially the same as “detecting invasive tumor cells” in the colonic-mucous-layer detachment fluid of the present invention, such terms may also be used interchangeably.

In another embodiment of the present invention, an detection method of invasive tumor cells known in the art other than detecting disease-related markers, or a diagnostic method of degree of malignancy and degree of invasion of tumors may be used. Examples of such detection method or diagnostic method of degree of malignancy and degree of invasion include, but are not limited to, for example, biopsy and a method of measuring tumor size. Such method may be used alone as a detection method of tumor cells, but may be used in combination with the above detection method of disease-related markers. By using these methods in combination, more accurate detection of invasive colorectal tumors becomes possible. The present invention further relates to a method for detecting disease-related markers, comprising a step of extracting nucleic acid from the specimen of the present invention.

In the method of the present invention, any publicly-known methods may be used for extracting disease-related markers from a specimen. Typically, a specimen is centrifuged, and the pellet obtained is re-suspended in an appropriate medium such as PBS and saline, digested with a protein dissolving agent such as proteinase K, deproteinized by an organic solvent such as phenol and chloroform, and nucleic acids are precipitated by ethanol, etc. to extract the nucleic acids. Concrete protocols are described in various references regarding genetic engineering (for example, Chomczynski, P., Sacchi, N.: Anal. Biochem., 162: 156-159, 1987; Masami Muramatsu and Masashi Yamamoto, Ed., New Handbook of Genetic Engineering, 4th revision, Yodosha, Oct. 2003, 20-29), and therefore they are not described in detail here.

It is preferable that the specimen is subjected to extraction treatment immediately after its collection; however, the specimen can be stored for a certain period before extraction treatment, for example approximately 12 hr, and also approximately 24 hr. Preferred storage temperature is, from the viewpoint of protection of disease-related markers, preferably −80° C. to 20° C., more preferably −80° C. to 10° C., and particularly preferably −80° C. to 4° C. When a specimen is to be stored, preferably it is stored in a re-suspended state in the above appropriate medium after centrifugation.

The extracted nucleic acid can be detected by various detection methods corresponding to the target disease markers, including for example, various nucleic acid amplification methods such as PCR, nucleic acid sequence-based amplification (NASBA), transcription mediated amplification (TMA), ligase chain reaction (LCR), strand displacement amplification (SDA), loop-mediated isothermal amplification (LAMP), isothermal and chimeric primer-initiated amplification of nucleic acids (ICAN), and branched DNA, as well as southern blotting, northern blotting, RNase protection assay, microarray, dot blot, and slot blot, etc.

In particular, when the marker is epigenetic methylated DNA, it can be detected by, for example, bisulfite sequencing method, methylation-specific PCR (MS-PCR), combined bisulfite restriction assay (COBRA), MS-SNuPE, bisulfite-SSCP, differential methylation hybridization (DMH), methyLight method, pyrosequencing, etc.

In addition, quantification of nucleic acids may be carried out using any known method including spectrophotometric method for measuring absorbance at maximum absorption wavelength of approximately 260 nm, and a method using various reagents that stain nucleic acids such as ethidium bromide, DAPI (4,6-diamidine-2-phenylindole), acridine orange, Mupid (registered trademark)-STAIN eye (Advance Co., Ltd.), diphenylamine reagent, Hoechst 33258 (H33258), Quant-iT PicoGreen dsDNA Reagent (Invitrogen), Quant-iT RiboGreen (registered trademark) RNA Reagent (Invitrogen), Gel Indicator RNA Staining Solution (Funakoshi), SYBR (registered trademark) Green I or II, SYBR (registered trademark) Gold, and GelRed, etc.[0057]

In one preferred embodiment of the present invention, detection is performed by spraying a washing fluid onto the colonic mucous layer that includes a site of lesion, and using the collected colonic-mucous-layer detachment fluid as a specimen. In the colonic-mucous-layer detachment fluid of the present invention, as mentioned above, the mucous layer detached from the site of lesion comprises many exfoliated tumor cells, and it has been found that the higher the degree of invasion of the site of lesion, i.e., the tumor, the larger the number of said tumor cells are comprised. Therefore, it is possible to diagnose whether or not the tumor is invasive, i.e., whether or not the site of lesion is malignant, by collecting the washing fluid sprayed on the site of lesion and using said fluid as a colonic-mucous-layer detachment fluid, and by examining the cells in said detachment fluid. Namely, this means that when an invasive tumor is detected, then such site of lesion can be determined to be malignant. Whether the site onto which a washing fluid is to be sprayed is a site of lesion or not, may be determined by determination method of site of lesion known in the art, such as macroscopic observation using an endoscopy.

As mentioned above, the detection method of the present invention can determine whether a site of lesion is malignant, or whether a tumor is invasive. Accordingly, it is preferable that the site of lesion is a site of lesion where tumor invasion is suspected.

As described above, any disease-related markers known in the art which can detect invasive colorectal tumors can be used as the disease-related markers used in the present invention. Since the colonic-mucous-layer detachment fluid of the present invention does not substantially comprise components derived from the digestive tract other than the large intestine, the detected disease-related markers are judged to be substantially derived from colorectal tumors. Accordingly, the disease-related marker that can be used in the present invention is a disease-related marker of invasive colorectal tumors, i.e., colorectal cancer. Since the mucous-layer detachment fluid of the present invention comprises tumor cells themselves, secretory disease-related markers such as secretory proteins and hormones are not suitable for use in the present invention as disease-related markers. Therefore, more preferably, examples include disease-related markers that can be detected by collecting cells, such as grade of cellular atypism obtained by morphological observation by cytological diagnosis, as well as detection of oncogenes or mutated tumor suppressor genes and abnormally modified DNA. Of these, a more preferable example is mutated K-RAS because mutation site relating to the progress of cancer has been identified. In the present invention, the term “detection of mutated K-RAS” is used exchangeable with the term “detection of K-RAS mutation.” In addition, when “K-RAS mutation” is simply referred, it includes both mutation of K-RAS genes and mutation of K-RAS proteins caused thereby.

Examples of another more preferable disease-related marker include detection of abnormally methylated DNA, due to reasons that it is detectable from only a minute amount of DNA, and that it is inherited to daughter cells with high conservative property.

In other preferable embodiment, determination of disease-related marker levels is performed by cytological diagnosis, because it is easy to carry out.

In the present invention, “modified DNA” refers to DNA, wherein a base or a sugar chain of a nucleotide that constitutes the DNA is modified by a modifying group. Examples of such modifying group include, typically, a methyl group.

In the present invention, “methylation of DNA” or “DNA methylation” refers to that one or more nucleotides contained in a DNA sequence are methylated. Typically, the base cytosine is methylated to form 5-methylcytosine. In the present invention, DNA that is subjected to DNA methylation is referred to as “methylated DNA.” Especially, DNA methylation is known to be deeply involved in the transcription activity of genes.

In the present invention, “abnormally modified DNA” means a state in which modified DNA is excessively increased or decreased, compared to normal cells. In particular, when the modified DNA is methylated DNA, the modified DNA significantly affects gene expression profile in a cell by affecting the transcription activity of the gene.

In the present invention, “methylation of CpG island” refers to that a CpG island present in the promoter region of any genomic gene is methylated. Typically, cytosine of CpG is methylated. It is known that by hypermethylation of CpG islands, silencing of genes downstream of the CpG islands occurs. Then, in cancer, it is considered that, for example, silencing of tumor suppressor genes is induced by hypermethylation of CpG islands, resulting in progress of canceration of cells.

“DNA methylation level” refers to a ratio of methylated nucleotides to all nucleotides that can possibly be methylated in a given gene. In particular, when referring to “methylation level in the promoter CpG island regions of a gene,” it means a ratio of methylated cytosines relative to the cytosines in all CpG sequences existing in said promoter region.

Therefore, in one preferred embodiment of the present invention, methylated DNA is adopted as the disease-related marker. Abnormal methylation of DNA is known to play a function in canceration. Canceration is considered to occur as follows, for example: by hypermethylation in the promoter CpG island regions upstream of a tumor suppressor gene as mentioned above, canceration may progress due to silencing of tumor suppressor genes, or canceration may occur by instable genome due to low-methylation state of the entire genome. Accordingly, by measuring the level of DNA methylation in a cell, presence of invasive tumors can be detected.

Ina more preferred embodiment of the present invention, the methylated DNA is methylation of a CpG island. Particularly preferred is the methylation in the promoter CpG island regions of one or more genes selected from the group consisting SFRP1, SFRP2, DKK2 and hsa-mir-34b/c, namely, the genes wherein the CpG islands in their promoter region are known to be excessively methylated in colorectal cancer.

In another preferred embodiment of the present invention, K-RAS mutation is detected as the disease-related marker. K-RAS is a protein that acts in the signaling pathway for cell growth from EGFR to the nucleus, and specific mutation of K-RAS is known to promote canceration by constantly inducing such signaling. Therefore, by detecting presence/absence of K-RAS mutation, it is possible to determine the presence of invasive tumors.

The method of the present invention for obtaining an index to detect invasive colorectal tumors may comprise a step of spraying a washing fluid onto the colonic mucous layer to detach the mucus from the mucous layer, thereby collecting a specimen comprising the detached colonic mucus in said washing fluid, a step of extracting a nucleic acid in the specimen, and a step of determining the presence or absence of a disease-related marker in the nucleic acid.

In the present invention, regarding the criteria for determining the presence of invasive colorectal tumors using DNA methylation level of SFRP1 gene as the index, preferably, presence of an invasive colorectal tumor can be determined when the DNA methylation level in the promoter region of SFRP1 gene is more than 45%, and more preferably, presence of an invasive colorectal tumor can be determined when the DNA methylation level in the promoter region of SFRP1 gene is more than 51%.

In the present invention, regarding the criteria for determining the presence of invasive colorectal tumors using DNA methylation level of SFRP2 gene as the index, preferably, presence of an invasive colorectal tumor can be determined when the DNA methylation level in the promoter region of SFRP2 gene is more than 10%, and more preferably, presence of an invasive colorectal tumor can be determined when the DNA methylation level in the promoter region of SFRP2 gene is more than 33%.

In the present invention, regarding the criteria for determining the presence of invasive colorectal tumors using DNA methylation level of DKK2 gene as the index, preferably, presence of an invasive colorectal tumor can be determined when the DNA methylation level in the promoter region of DKK2 gene is more than 10%, and more preferably, presence of an invasive colorectal tumor can be determined when the DNA methylation level in the promoter region of DKK2 gene is more than 11%.

In the present invention, regarding the criteria for determining the presence of invasive colorectal tumors using DNA methylation level of mir-34b/c gene as the index, preferably, presence of an invasive colorectal tumor can be determined when the DNA methylation level in the promoter region of mir-34b/c gene is more than 15%, and more preferably, presence of an invasive colorectal tumor can be determined when the DNA methylation level in the promoter region of mir-34b/c gene is more than 18%.

Furthermore, more accurate detection of invasive colorectal tumors can be carried out by determining marker levels of a plurality of disease-related markers in the specimen of the present invention. Accordingly, the present invention comprises, in its one preferred embodiment, determining two or more disease-related marker levels. Any combination of two or more disease-related markers maybe used, and examples include, but are not limited to, a combination of different types of markers, such as a combination of cytological diagnosis with determination of DNA methylation level, or a combination of detection of K-RAS mutation with determination of DNA methylation level, as well as a combination of the same types of markers, such as a combination of methylation level in the promoter CpG island regions of SFRP1 with methylation level in the promoter CpG island regions of hsa-mir-34b/c.

As a determination manner for the above combinations, the following manner may be adopted: any two or more markers are selected from a plurality of markers, and when judgment criteria for determining presence of invasive colorectal tumors are satisfied in all the selected markers, then the presence of an invasive colorectal tumor is determined. As an example of this case, three disease-related marker levels are determined, then any two markers are selected; when judgment criteria are satisfied in both of the two markers, it is determined that an invasive colorectal tumor is present.

In addition, presence/absence of invasive colorectal tumors may be determined by a flow-chart manner by making the judgment criteria of each marker as branch conditions.

As mentioned above, it is also possible to use a combination of detection of invasive colorectal tumors by determination of disease-related marker levels with detection of invasive colorectal tumors by a means other than the detection of disease-related markers. For example, classification by tumor size or by cytological diagnosis may be combined with, for example, determination of disease-related marker levels such as detection of K-RAS mutation or determination of DNA methylation level.

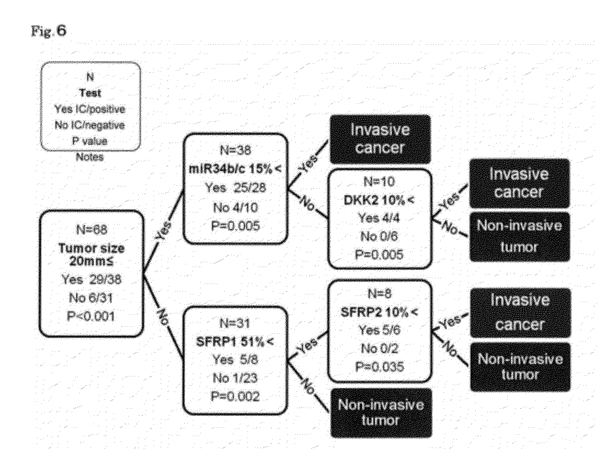

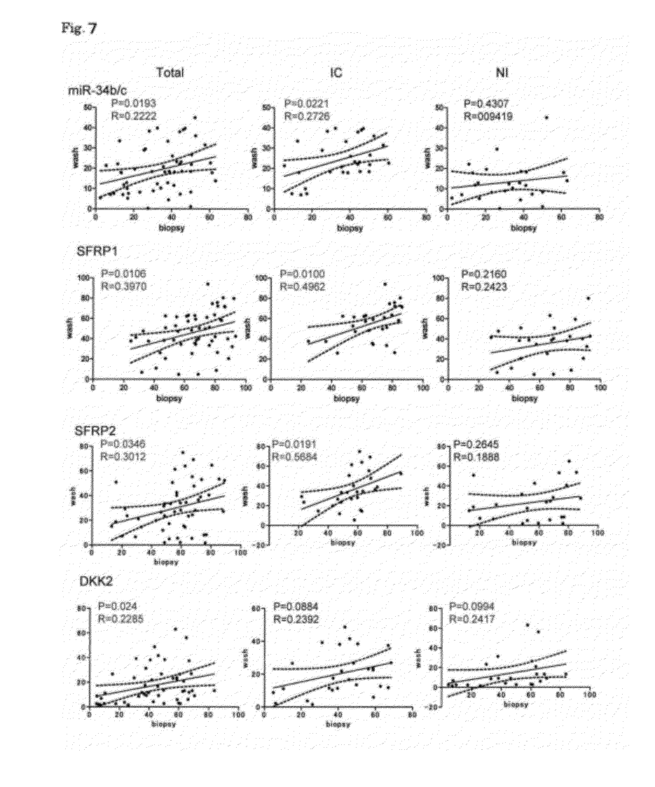

In FIGS. 6 and 10, an example of a diagnosis tree to determine presence or absence of an invasive tumor in a flow-chart manner is shown, wherein the tumor size is combined with determination of a plurality of DNA methylation levels.

For example, regarding the tumor size, when the diameter of a tumor observed from the large intestinal lumen side is 20 mm or larger, it can be determined that an invasive colorectal tumor is present. Furthermore, for example, when the diameter of a tumor observed from the large intestinal lumen side is 20 mm or larger and the DNA methylation level in the promoter region of hsa-mir-34b/c gene is more than 15%, it can be determined that an invasive colorectal tumor is present. In another embodiment, when the diameter of a tumor observed from the large intestinal lumen side is 20 mm or larger and the DNA methylation level in the promoter region of hsa-mir-34b/c gene is 15% or less and the DNA methylation level in the promoter region of DKK2 gene is more than 10%, it can be determined that an invasive colorectal tumor is present. Moreover, in another embodiment, when the diameter of a tumor observed from the large intestinal lumen side is 20 mm or larger and the DNA methylation level in the promoter region of SFPR1 gene is more than 51% and the DNA methylation level in the promoter region of SFPR2 gene is more than 10%, it can be determined that an invasive colorectal tumor is present.

In the detection method of the present invention, it is possible to diagnose the degree of invasion of invasive colorectal tumors by appropriate selection of disease-related markers and appropriate setting of threshold values. Accordingly, in other embodiment of the present invention, a method for diagnosing the degree of invasion of an invasive colorectal tumor, comprising: (a) determining one or more disease-related marker levels in the colonic-mucous-layer detachment fluid of the present invention, and (b) determining the degree of invasion based on the disease-related marker levels, is also encompassed by the present invention.

In the present invention, “degree of invasion” represents a degree of invasion of an invasive tumor from the lumen side to the abdominal cavity side. The degree of invasion maybe expressed based on a conventionally-used classification method, or may be expressed by an original standard that is suitable for use in the present invention.

For example, it has been found by the present inventors that the methylation level in the promoter CpG island regions of a tumor suppressor gene in the colonic-mucous-layer detachment fluid increases as the degree of invasion of the tumor existing at the spraying site of the washing fluid increases. Accordingly, it is preferable that the degree of invasion of invasive colorectal tumors is diagnosed by determining the methylation level in the promoter CpG island regions of a tumor suppressor gene. Examples of the tumor suppressor gene used in such embodiment include, but are not limited to, SFRP1 and hsa-mir-34b/c, etc. In the present embodiment, similar to the detection of invasive colorectal tumors, a combination of a plurality of disease-related markers may be used, and a combination with other indices related to degrees of invasion may be used. The diagnosis tree shown in FIGS. 6 and 10 may also be used in the diagnosis of the present embodiment.

The method of the present invention provides a remarkable result, considering the fact that in conventional biopsy, no significant difference in the methylation levels was observed between invasive tumors and non-invasive tumors; such remarkable results can be for the first time achieved by using the colonic-mucous-layer detachment fluid of the present invention. The reason that only in the colonic-mucous-layer detachment fluid of the present invention, significant differences are observed between invasive tumors and non-invasive tumors has not yet been clarified; however, it is considered as follows: as the degree of invasion increases, the number of tumor cells mixed in the colonic-mucous-layer detachment fluid of the present invention increases as mentioned above, and therefore, the ratio of DNA derived from tumor cells relative to the entire DNA obtained from the colonic-mucous-layer detachment fluid of the present invention increases.

Degrees of invasion of colorectal tumors may be classified using classification methods known in the art, for example TNM classification, classification on the basis of Japanese Classification of Colorectal Carcinoma, Dukes classification, etc. In this specification, the term “invasion depth” is used in cases where a degree of invasion is expressed in accordance with a conventionally-used classification method in the art, and accordingly, it is within the range of “degree of invasion.” For example, in TNM classification, invasion depth of primary colorectal tumors is classified as follows.

Tis: Carcinoma in situ: intraepithelial or invasion of lamina propria
T1: Tumor invades submucosa
T2: Tumor invades muscularis propria
T3: Tumor invades through the muscularis propria into the pericolorectal tissues
T4a: Tumor penetrates to the surface of the visceral peritoneum
T4b: Tumor directly invades or is adherent to other organs or structures

Examples of a diagnostic method of degrees of invasion include, for example, giving a score for degree of invasion that corresponds to the level of a selected disease-related marker. For example, when the disease-related marker is methylation in the promoter CpG island regions of SFRP1, and when the methylation level is for example 40.0% or less, preferably 35.0% or less, then the invasion depth is diagnosed to be Tis; when the methylation level is for example 50.0% or more, preferably 55.0% or more, then the invasion depth is diagnosed to be T2 or deeper. For instance, when the disease-related marker is methylation in the promoter CpG island regions of hsa-mir-34b/c, and when the methylation level is for example 20.0% or less, preferably 16.0% or less, then the invasion depth is diagnosed to be Tis; when the methylation level is for example 25.0% or more, preferably 28.0% or more, then the invasion depth is diagnosed to be T2 or deeper.

Furthermore, the method for detecting invasive colorectal tumors of the present invention may also be used as a method for evaluating therapeutic effects of a drug and/or a therapeutic method.

For example, by comparing the marker level A prior to treatment with the marker level B after treatment, or by comparing the marker level A at a certain time point during treatment with the marker level B at other time point after the previous time point during treatment, and when A>B, then it can be evaluated that therapeutic effects are observed; when A<B, then it can be evaluated that no therapeutic effects are observed, and when A=B, then it can be evaluated that therapeutic effects are unknown.

The method of the invention can also be used for monitoring presence/absence of recurrence of the disease after completion of treatment. In this case, a specimen is collected from a subject at regular intervals after completion of treatment, and presence/absence of the disease is checked by the same method as described above based on marker levels.

In one aspect of the present invention, a kit for collecting a colonic-mucous-layer detachment fluid, containing at least one sealable specimen collection container for collecting the above-mentioned sprayed washing fluid and a preservative solution, is provided. The specimen collection container contained in the kit of the present invention may be those of a detachable structure to a conventional washing-fluid spraying device, or may be those integrated into a washing-fluid spraying device; those that can be detachably attached are preferable. The preservative solution is a liquid that can store collected colonic-mucous-layer detachment fluid in a manner that can be used afterwards, such as cell preservative solutions and DNA preservative solutions. Preferably, the preservative solution comprises SDS, EDTA or Tris, etc., and has effects to denature nucleolytic enzymes and to suppress function of nucleolytic enzymes by chelating specific ions. Such preservative solutions are commercially available and may be used. Examples of commercially available preservative solutions include Thinprep pap test from Cytyc corporation.

The kit of the present invention may further contain an operating part in an aspiration tube, a connector, an aspiration tank, an aspirator, a spraying tube, or a part therebetween. In addition, these may be those detachably attached to the above specimen collection container, or they may be formed with the specimen collection container in an integrated manner.

The kit of the present invention may further contain various tools used for treating collected specimens. Examples of such components include a tool for fixing a specimen (substance and/or apparatus), a tool for staining a specimen, a tool for extracting nucleic acid and/or protein from a specimen, a tool for determining disease-related marker levels in the nucleic acid and/or protein in a specimen or extracted from the specimen. Regarding these tools, those usually used for such purposes in the art may be used.

Various methods are known as the detection method of methylated DNA; from the viewpoint of quantitative characteristic, etc., a pyrosequencing method is preferably used. Therefore, in one preferred embodiment of the kit of the present invention, the kit further comprises one or more primers used for detecting methylated DNA, as a tool to determine disease-related marker levels. More preferably, the primer is a primer used for detecting methylation in the promoter CpG island regions of one or more genes selected from the group consisting of SFRP1, SFRP2, DKK2 and hsa-mir-34b/c.

Unless otherwise stated in this specification, scientific and technical terms used in relation to the present invention shall have the meaning that is usually understood in the art. In general, as used herein, terms and technologies used with respect to cells and tissue cultures, molecular biology, immunology, microbiology, gene and protein and nucleic acid chemistry shall be those well-known and commonly used in the art. In addition, unless otherwise stated, the methods and technologies of the present invention are carried out in accordance with well-known conventional methods in the art as described in various general and specialized references cited and discussed herein. Examples of such references include Sambrook et al., Molecular Cloning: A Laboratory Manual 2^{nd} ed , Cold Spring Harbor Laboratory Press (1989) and Sambrook et al., Molecular Cloning: A Laboratory Manual, 3^{rd} ed., Cold Spring Harbor Press (2001); Ausubel et al., Current Protocols in Molecular Biology, Greene Publishing Associates (supplements in 1992 and 2000); Ausubel et al., Short Protocols in Molecular Biology: A Compendium of Methods from Current Protocols in Molecular Biology-4th Ed., Wiley & Sons (1999); Harlow and Lane, Antibodies: A Laboratory Manual, Cold Spring Harbor Laboratory Press (1990); and Harlow and Lane, Using Antibodies: A Laboratory Manual, Cold Spring Harbor Laboratory Press (1999).

Terms used with respect to analytical chemistry, synthetic organic chemistry and medicinal chemistry as described herein, and the experimental procedures and techniques thereof are those that are well known and normally used in the art. Standard techniques are used in chemical synthesis, chemical analysis, preparation, formulation and delivery of drugs, and treatment of subjects.

As used herein, unless otherwise stated, the name of a protein is expressed by an alphabetical (or alphanumeric) name of the gene followed by “protein,” and the gene encoding the protein is expressed by the above alphabetical (or alphanumeric) name of the gene alone, or by the alphabetical (or alphanumeric) name of the gene followed by “gene.” The above “gene” means any one of the genomic gene, messenger RNA or cDNA; when any one of them is to be specified, it should be specifically expressed.

In the present invention, “subject” means any individual organism having the large intestine, and is preferably a mammal such as humans, nonhuman primates, companion animals such as dogs and cats, as well as industrial animals such as cows, horses, goats, sheep and pigs, and is particularly preferably a human. In the present invention, the subject may be a healthy subject, or may be suffering from a certain disease, or may be under treatment or after treatment of disease.

In the present invention, “tumor” refers to a condition of abnormal cell growth, and among tumors, malignant tumors are referred to as “cancer.”

In the present invention, “invasive colorectal tumor detection” or “detection of an invasive colorectal tumor” is equivalent to detecting the presence of an invasive colorectal tumor, and it is one embodiment of “diagnosis of invasive colorectal tumors.” Its examples may include, but are not limited to, a method for obtaining an index used to detect whether or not an invasive colorectal tumor is present.

As used herein, “epigenetic” shall have a usual meaning in the art of the present invention, and is exemplified by, for example, that gene expression is regulated by an acquired modification to chromatin without changes in the DNA sequence.

Examples of acquired modification to chromatin include, in addition to methylation of DNA bases, chemical modification such as methylation, acetylation, and phosphorylation of histone.

In the present invention, “invasive tumor” refers to a tumor that infiltrates through the muscular layer of the mucosa into the submucosa or deeper layers.

In the present invention, “non-invasive tumor” refers to a tumor that is confined to the mucosa.

In the present invention, “noncancer” is used to refer to a condition that is not cancerous. Accordingly, noncancer condition includes healthy states as well as tumors that are not cancerous.

In the present invention, “physiological isotonic solution” refers to an isotonic solution with nearly neutral pH, which does not comprise a component that irritates the colonic mucosa.

In the present invention, “hsa-mir-34b/c,” “mir-34b/c” and “microRNA34b/c” encompass a meaning of general microRNA, as well as a genomic gene common to micro RNA 34b and micro RNA 34c. When the term “hsa-mir-34b/c” or “mir-34b/c gene” is used, it means particularly explicitly a genomic gene.

In the present invention, in the representation of a primer sequence used for detecting methylated DNA, the base represented by “Y” means thymine (T) when unmethylated cytosine of CpG islands is to be detected, and it means cytosine (C) when methylated cytosine of CpG islands is to be detected.

In the present invention, in the representation of a primer sequence used for detecting methylated DNA, the base represented by “R” means adenine (A) or guanine (G), so that primers wherein “R” is adenine (A) and primers wherein “R” is guanine (G) are used in a mixture.

In the present invention, “ROC curve”refers to a receiver operating characteristic curve.

The ROC curve can be used for determining a threshold value to judge positive result or negative result, when a specimen is to be tested positive/negative for a specific disease, based on test results expressed by continuous numerical values.

In the ROC curve, “sensitivity” indicates the ratio of specimens that were correctly tested positive which must be tested positive, and “specificity” indicates the ratio of specimens that were correctly tested negative which must be tested negative. In general, when a specimen that must be tested positive was correctly tested positive, this is defined as “true positive;” when a specimen that must be tested positive was erroneously tested negative, this is defined as “false-negative;” when a specimen that must be tested negative was correctly tested negative, this is defined as “true negative;” when a specimen that must be tested negative was erroneously tested positive, this is defined as “false-positive.” The “sensitivity” can be calculated by dividing the number of true positive by the summation of the number of true positive and the number of false-negative. In addition, the “specificity” can be calculated by dividing the number of true negative by the summation of the number of false-positive and the number of true negative.

“AUC” indicates the area under a ROC curve. “AUC” is a value between 0.5 and 1.0, and as the value approaches 1.0, this indicates that the test method is superior.

“Best cut-off,” that is the most appropriate threshold value to differentiate invasive tumor from non-invasive tumor, can be obtained as the point on the ROC curve located at the shortest distance from the upper-left point on the ROC curve at which “sensitivity” is 1 and “1-specificity” is 0.

TABLE 1
Characteristic features of subjects from which specimens were collected.

| | Hyperplastic | | Early | Advanced |
|---|---|---|---|---|
| Tumor | polyp | Adenoma | cancer | cancer |
| Age (mean ± SD) | 63.2 | 66.047 | 68.21 | 67.27 |
| Male | 5 | 52 | 15 | 32 |
| Female | 5 | 11 | 4 | 16 |
| Endoscopic findings | | | | |
| Protruded type | 0 | 32 | 7 | |
| Flat type | 10 | 29 | 7 | |
| Depressed type | 0 | 1 | 3 | |
| Bormann type 1 | | | | 7 |
| Bormann type 2 | | | | 31 |
| Bormann type 3 | | | | 6 |
| Bormann type 4 | | | | 0 |
| Others | | | | 4 |
| Histologic grade | | | | |
| Mild-moderate | | 51 | | |
| dysplasia | | | | |
| Severe dysplasia | | 5 | | |
| Differentiation | | | | |
| Well to | | | 19 | 45 |
| moderately | | | | |
| differentiated | | | | |
| Undifferentiated | | | 0 | 1 |
| (poorly differentiated | | | | |
| adenoma and signet | | | | |
| ring cell carcinoma) | | | | |
| Tumor size | | | | |
| <20 mm | 10 | 57 | 2 | 0 |
| 20 mm ≦ | 0 | 6 | 17 | 46 |
| Clinical stage | | | | |
| Stage 0 | | | 12 | 0 |
| Stage 1 | | | 5 | 8 |
| Stage 2 | | | 2 | 32 |
| Stage 3 | | | 0 | 5 |
| Stage 4 | | | 0 | 1 |

TABLE 2
Comparison of cases and DNA contents between specimens obtained
by Method 1 and specimens obtained by Method 2.

| | No. of cases | | | | | | | |
|---|---|---|---|---|---|---|---|---|
| | 11 | | | | 37 | | | |
| | | Colonic-mucous-layer | | | | Colonic-mucous-layer | | |
| | | detachment fluid (Method 1) | | | | detachment fluid (Method 2) | | |
| | | | Invasive | Non-invasive | | | Invasive | Non-Invasive |
| | Biopsy | Total | tumor | tumor | Biopsy | Total | tumor | tumor |
| Amount of | 21.28 ± 15.52 | 15 ± 11.12 | | | 17.08 ± 13.0 | 16.53 ± 16.88 | | |
| DNA | | | | | | | | |
| No. of | | 2 | 1 | 1 | | 17 | 11 | 6 |
| diagnosed | | | | | | | | |
| cases | | | | | | | | |
| No. of not | | 9 | 7 | 2 | | 20 | 7 | 13 |
| diagnosed | | | | | | | | |
| cases | | | | | | | | |
| Ratio of | | 18.2% | 12.5% | 33.3% | | 45.9% | 31.6% | 61.1% |
| diagnosed | | | | | | | | |
| cases | | | | | | | | |

TABLE 3
Comparison between cases of biopsy sample and cases of colonic-
mucous-layer detachment fluid collected by Method 2.

| | | | | | | Colonic-mucous-layer | | | |
|---|---|---|---|---|---|---|---|---|---|
| | | Biopsy | | | | detachment fluid (Method 2) | | | |
| | | Invasive | Non-Invasive | Sig. | Normal | Invasive | Non-Invasive | Sig. | Normal |
| | | tumor | tumor | dif. | tissue | tumor | tumor | dif. | tissue |
| No. of cases | | 52 | 98 | | 187 | 36 | 34 | | 6 |
| Ave. age | | 67.4 | 66.7 | None | 67.2 | 68 | 63.97 | None | 61.67 |
| Sex | Male | 16 | 24 | None | 126 | 22 | 25 | None | 3 |
| | Female | 36 | 74 | None | 61 | 14 | 9 | None | 3 |
| Tumor size | <20 mm | 9 | 77 | | | 7 | 17 | | |
| | 20 mm< | 43 | 21 | | | 29 | 8 | | |
| Histological | Hyper-plastic or | | 15 | | | | 3 | | |
| findings | inflammatory | | | | | | | | |
| | Tubular adenoma | | 29 | | | | 10 | | |
| | Ductal | | 28 | | | | 7 | | |
| | chorioadenoma | | | | | | | | |
| | Advanced | | 26 | | | | 14 | | |
| | dysplasia | | | | | | | | |
| | Cancer | 52 | | | | 36 | | | |

TABLE 4
Detection rate of K-RAS mutation.

| | | Non-invasive | |
|---|---|---|---|
| | Invasive tumor | tumor | All specimens |
| Biopsy sample | 9 | 6 | 14 |
| Mucous | 7 | 2 | 8 |
| detachment | | | |
| fluid (Method 2) | | | |
| Concordance | 77.7% | 33% | 62% |
| ratio | | | |

### EXAMPLES

Hereafter, the present invention is described more specifically; however, the invention is not limited to the following examples. In addition, in the following examples, “methylated DNA of a gene” means methylation in the promoter CpG is land regions located upstream of said gene, unless stated otherwise.

### Example 1

### Investigation of Collection Method of Specimens

The specimens were collected during November 2008 and June 2009 in the Digestive Organs and Endoscopy Center of the Akita Red Cross Hospital. The subjects from which the specimens were collected include patients who have had colonic adenomas for a long time and who took colorectal endoscopy for follow-up purposes, or patients who took the same for the purpose of pre-operation of colorectal cancer surgery, or those who took the same for detailed examination by reason of positive results of fecal occult blood tests in check-ups. Biopsy was carried out for 52 specimens of invasive tumor, 98 specimens of non-invasive tumor, and 187 specimens of normal mucosa. Mucous-layer detachment fluid was collected for 34 specimens of invasive tumor, 36 specimens of non-invasive tumor, and 6 specimens of normal mucosa. Table 1 shows characteristics of subjects from which specimens were collected.

“Hyperplastic polyp,” “adenoma,” “early cancer,” and “advanced cancer” were determined by histopathological diagnosis. Classification of protruded type, flat type, depressed type, Bormann type 1, Bormann type 2, Bormann type 3 and Bormann type 4 was in accordance with the “General Rules of Colorectal Cancer”. Degree of dysplasia and degree of differentiation were determined in accordance with histopathological diagnosis. Tumor size was determined by measuring pathological specimens. Determination of clinical stage was in accordance with the General Rule and TNM classification.

In the subsequent experiment, statistical analysis was performed using 12 specimens of normal mucosa (6 males and 6 females) in addition to the group of mucous-layer detachment fluid, but no difference in the results was observed compared to those shown below.

Furthermore, several months after the above period, additional 41 specimens of biopsy and 47 specimens of mucous-layer detachment fluid were examined as the test set. Here, in order to differentiate the specimens of the test set from the present test group, the test group comprising the above 337 biopsy specimens and 88 mucous-layer detachment fluid specimens is referred to as the training set.

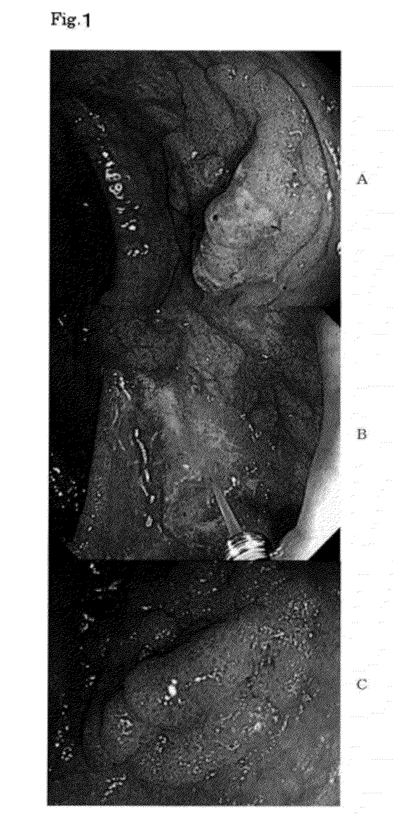

In order to investigate collection method of specimens, specimens were collected by either of the following two methods: (Method 1) for 13 cases of colorectal cancer, tap water was sprayed onto the tumor site at a rate of 5 ml/s by a colon fiberscope using directly a 50-ml syringe barrel, and 10 ml of the water comprising detached mucous layer was collected; (Method 2) for 46 cases of colorectal cancer, 20 ml of saline was sprayed onto the tumor site at a rate of 5 ml/s by a Pyoktanin-spraying tube (NT tube, Olympus), and 10 ml of the water comprising detached mucous layer was collected. The number of subjects from which specimens were collected by Method 1 was 11 cases, and the number of subjects from which specimens were collected by Method 2 was 37 cases. FIG. 1 shows a photograph of the lumen of the large intestine before spraying saline onto the tumor site using a Pyoktanin-spraying tube (FIG. 1A), a photograph of the lumen of the large intestine during spraying (FIG. 1B), and a photograph of the lumen of the large intestine after spraying (FIG. 1C).

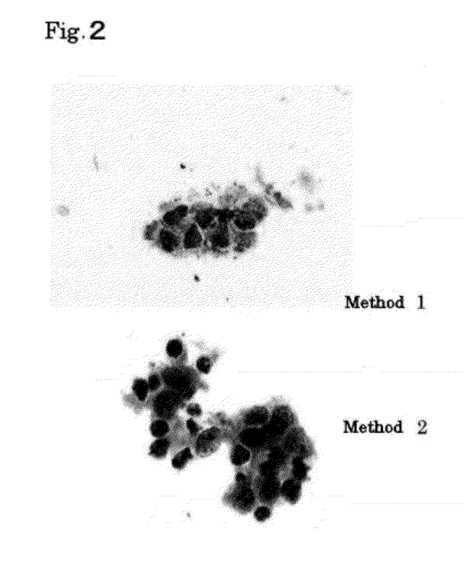

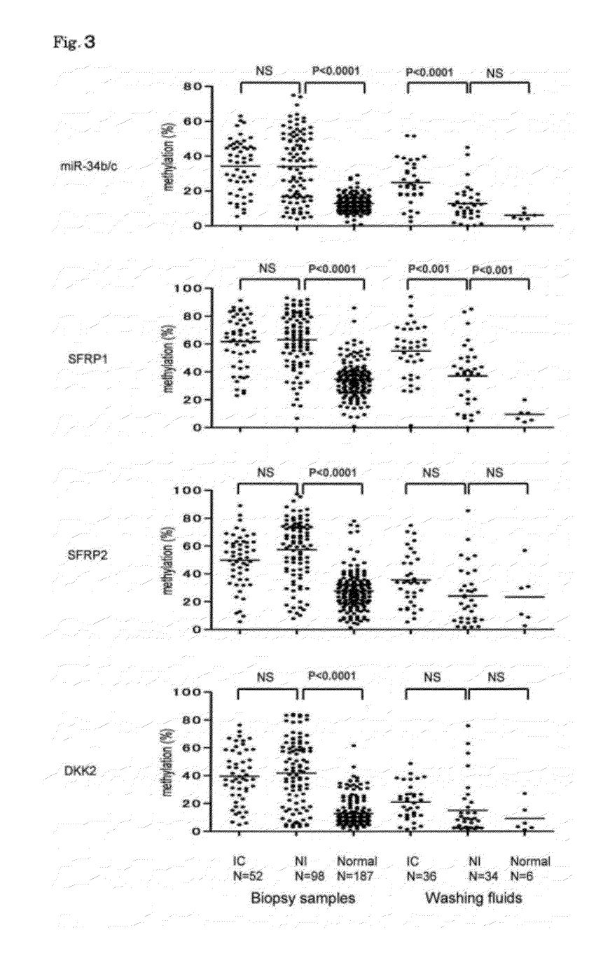

The obtained specimens were centrifuged at 1500 rpm for 10 min, cells were precipitated, supernatant was discarded, and apart of the cells were formalin-fixated and stained with hematoxylin-eosin, then morphology of the cells was observed. Cytological diagnosis of colonic-mucous-layer detachment fluid of the large intestine (21 cases of advanced cancer, 5 cases of early cancer, 15 cases of adenoma) was carried out and pathological diagnosis of colorectal tumors was compared with the cytological diagnosis of colonic-mucous-layer detachment fluids. The results of the biopsy agreed with those of cytological diagnosis in 10 cases of advanced cancer (concordance rate: 10%), 0 cases of early cancer (concordance rate: 0%), 4 cases of adenoma (concordance rate: 26.6%). These results suggest that colorectal cancer cells are floating in the colonic-mucous-layer detachment fluid, and in particular, tumor cells are markedly contained in the mucosa detachment fluids from advanced colorectal cancer. FIG. 2 shows photographs of cells in the specimens collected by either Method 1 or Method 2. In Method 1, the number of cells is small and denaturation of cells is strong. In Method 2, denaturation becomes weak and a case was observed which could be diagnosed as adenocarcinoma by cytological diagnosis. In addition, cytological diagnosis of some of the cases exhibited an image wherein the number of cells was small and the degree of denaturation of cells was large by Method 1, and an image wherein the number of cells was large and the degree of denaturation of cells was small by Method 2. Thus, it is considered that by Method 2, tumor cells of the mucous layer can be efficiently collected and abnormal methylation of tumor cells can be analyzed.

Furthermore, DNA was extracted from the above centrifuged specimens, and the amounts of DNA obtained were compared. As shown in Table 2, no large difference was observed in the DNA content between the specimens obtained by Method 1 and the specimens obtained by Method 2. The amounts of DNA in the specimens collected by Method 1 are 15.00±11.12 μg for colonic mucous layer detachment fluids, and 21.28±15.52 μg for biopsy samples. In contrast, the amounts of DNA in the specimens collected by Method 2 are 16 .53±16.88 μg for colonic mucous layer detachment fluids, and 17.08±13.00 μg for biopsy samples. No difference was observed in the amount of DNA obtained between Method 1 and Method 2, and no difference was observed in the amount of DNA obtained between the colonic-mucous-layer detachment fluids and the biopsy tissues.

Meanwhile, DNA extraction was performed as follows. After centrifugation of a specimen, supernatant was removed and the pellet was re-suspended in 4.5 ml of SEDTA. To this suspension, 0.5 ml of 10% SDS and 50 ml of 20 mg/ml proteinase K (TAKARA BIO INC., Code No. 9033) were added, and incubated at 55° C. for 1 hr. 5 ml of phenol (UltraPure Buffer-Saturated Phenol, Invitrogen Life Technologies) were added, and after mixing by rolling-over, the mixture was centrifuged at 2700 rpm and 4° C. for 15 min and the supernatant was transferred to a new tube. This procedure was repeated for additional 1-2 times, then the solvent was replaced with the same amount of chloroform (Wako Pure Chemical Industries, Ltd.) and the procedure was repeated for additional 1-2 times. To the resultant mixture, 5 ml of glycogen (Ambion Cat#9510) and 9 ml of 100% ethanol were added, and after mixing by rolling-over, the mixture was incubated at 4° C. for 12 hr . Then, the specimen was centrifuged at 4° C. for 15 min and supernatant was discarded, and the pellet was suspended in 10 ml of 70% ethanol, centrifuged at 2700 rpm and 4° C. for 15 min, supernatant was discarded, and the resulting substance was dissolved in 200 ml of purified water, giving a sample for DNA analysis.

With respect to the amount of DNA, the amount of collected DNA contained in a specimen was measured by spectrophotometric method. Spectrophotometric method was performed using a NanoDrop ND-1000 spectrophotometer (AGC Techno Glass Co., Ltd.) in accordance with the manufacturer's manual.

For the same subject, histopathological examination was performed, and when the invasion in or through submucosa was observed, it was diagnosed to be invasive tumor, and when noncancer was determined or cancer was limited to the mucosa, it was diagnosed to be non-invasive tumor. In addition, using the specimens obtained by Method 1, invasive tumor or non-invasive tumor was determined by histopathological analysis. Table 2 summarizes these results. With the specimens collected by Method 1, only 12.5% of invasive tumors and 33.3% of non-invasive tumors were diagnosed correctly, whereas with the specimens collected by Method 2, 31.6% of invasive tumors and 61.1% of non-invasive tumors were diagnosed correctly. Thus, it has been demonstrated that the specimens collected by Method 2 were superior to the specimens collected by Method 1, as specimens for detecting invasive colorectal cancer.

Furthermore, in order to compare biopsy samples with colonic-mucous-layer detachment fluid collected by Method 2, characteristic features of the cases from which specimens were obtained were shown in Table 3. No differences in the average age and numbers of males and females were observed between the cases from which biopsy samples were obtained and the cases from which colonic-mucous-layer detachment fluids were obtained by Method 2. In Table 3, determination of invasive tumor and non-invasive tumor was carried out by histopathlogical examination. From these results, no significant differences due to gender and age were observed. The average amounts of DNA obtained from the colonic-mucous-layer detachment fluids by Method 2 and from biopsy of colorectal tumors were 12.28±1.24 mg (N=87) and 15.86±0.633 mg (N=365), respectively, demonstrating that colonic-mucous-layer detachment fluid obtained by colonos copy comprises a sufficient amount of DNA for analysis.

### Example 2

### Measurement of DNA Methylation Level and Measurement of K-RAS Mutation

Next, DNA methylation level was measured for all cases. First, colonoscopy was performed and when a tumor was observed by colonoscopy of the entire large bowel, a washing fluid was sprayed by a Pyoktanin-spraying tube (NT tube, Olympus) from an adjacent position of the tumor to detach the mucus from the surface of the lumen of the large intestine. At this time, the washing fluid was sprayed onto the colonic mucous layer at a rate of approximately 5 ml/s.

Then, after observation by a magnified endoscope (CF-260AZI), background normal mucosae at the cancerous site of the tumor, adenoma site, and periphery of the tumor (within 1 cm) were collected by biopsy, and treated with EMR or marking, etc. The collected specimens were stored in endfresh, and after frozen storage, DNA extraction by phenol-chloroform method was performed.

The biopsy samples and colonic-mucous-layer detachment fluids collected were subjected to DNA extraction, then treated with sodium bisulfite using Epitect bisulfite kit from QIAGEN, in accordance with the protocol described in its handling manual “EpiTect (registered trademark) Bisulfite Protocol and Trouble Shooting” and “EpiTect (registered trademark) Bisulfite Handbook,” and PCR was carried out using 1 μl of this bisulfite DNA.

Regarding all the primers, primers for detecting methylated DNA of SFRP1, SFRP2, DKK2 and micro RNA 34b/c, for which methylation in colorectal cancer was reported, were used.

In concrete terms, for mir-34b/c gene, DNA with SEQ ID NO 1 was used as the forward primer, DNA with SEQ ID NO 2 was used as the reverse primer, and DNA with SEQ ID NO 3 was used as the pyrosequencing primer.

In addition, for SFRP1 gene, DNA with SEQ ID NO 4 was used as the forward primer, DNA with SEQ ID NO 5 was used as the reverse primer, and DNA with SEQ ID NO 6 or 7 was used as the pyrosequencing primer.

In addition, for SFRP2 gene, DNA with SEQ ID NO 8 was used as the forward primer, DNA with SEQ ID NO 9 was used as the reverse primer, and DNA with SEQ ID NO 10 was used as the pyrosequencing primer.

Similarly, for DKK2 gene, DNA with SEQ ID NO 11 was used as the forward primer, DNA with SEQ ID NO 12 was used as the reverse primer, and DNA with SEQ ID NO 13 or 14 was used as the pyrosequencing primer.

In all PCR assays, 50 cycles of the following steps were performed: a denaturation step at 95° C. for 30 s, then an annealing step at 60° C. for 30 s, and an extension step at 72° C. for 30 s. Thereafter, DNA methylation levels were quantitatively measured by a pyrosequencer, and an averaged value of respective methylation levels at the CpG site upstream of the primer was adopted.

With respect to mutation of K-RAS gene, codon 12 and codon 13 were measured using a pyrosequencing method.

Mutation of K-RAS gene was measured using a KRAS detection kit PyroMark KRAS v2.0 (4×24) (Catalogue No. 970452) from QIAGEN, in accordance with the protocol described in its handling manual “PyroMark (registered trademark) KRAS v2.0 Handbook.”

In the biopsy samples, methylation levels of SFRP1 , SFRP2, DKK2 and mir34b/c show tendencies of gradual increase in non-cancerous parts, adenoma and cancerous parts, but no significant difference was observed. In contrast, in the analysis of DNA methylation level using mucous-layer detachment fluid, in advanced cancers and in non-cancerous parts, DNA methylation level of SFRP1 is 52.5% in cancerous part and 11.0% in non-cancerous part, showing a significant difference with a significance level of P<0.001. The DNA methylation level of microRNA34b/c is 26.39% in cancerous part and 5.33% in non-cancerous part, showing a significant difference with a significance level of P<0.0007. Similarly, when early cancer is compared with non-cancerous part, the DNA methylation level of SFRP1 is 41.2% in early cancer and 11.0% in non-cancerous part, showing a significant difference with a significance level of p=0.0043. The DNA methylation level of microRNA34b/c is 23.38% in early cancer and 5.33% in non-cancerous part, showing a significant difference with a significance level of p=0.0220. When adenoma is compared with non-cancerous part, the DNA methylation level of SFRP1 is 32.6% in adenoma and 11.0% in non-cancerous part, showing a significant difference with a significance level of p=0.0089. The DNA methylation level of microRNA34b/c is 17.17% in adenoma and 5.33% in non-cancerous part, showing a significant difference with a significance level of p=0.0306. In addition, regarding the relationship between invasion depth of cancer and methylation, a significant difference in the methylation level was observed between the cases of invasion depth of m (cancer stays within the mucosa and does not invade submucosa, which corresponds to Tis in the TNM classification) and the cases of invasion depth of mp or deeper (cancer invades muscularis propia or deeper, which corresponds to T2 or deeper in the TNM classification) as follows: the DNA methylation level of SFRP1 was 33.39% for the invasion depth of m, and 57.17% for the invasion depth of mp, showing a significant difference with a significance level of P=0.0230. The DNA methylation level of microRNA34b/c was 15.62% for the invasion depth of m, and 29.36% for the invasion depth of mp, showing a significant difference with a significance level of P=0.0477. A correlation between the whole biopsy and the colonic-mucous-layer detachment fluid was observed only for the DNA methylation level of microRNA34b/c, with a significance level of P<0.001 and a correlation coefficient R of 0.4296±0.1000.

Presence/absence of K-RAS gene mutation was investigated using a part of specimens derived from the same lesion obtained as in Table 3, and the results are shown in Table 4. The results show that invasive tumors can be detected in 77.7% of the specimens using both of the biopsy samples and mucous detachment fluids collected by Method 2. Here, the total numbers of specimens do not agree, because in some cases, while K-RAS gene mutation was detected in biopsy, it was not detected in mucous detachment fluid. A large number of such cases were especially found for non-invasive tumors.

### Example 3

### Endoscopic Classification

Tumor morphology of hyperplastic polyp, adenoma, and early cancer was classified into protruded type (0-I), flat type (IIa, LST), or depressed type (IIc, IIa+IIc) by colorectal endoscopy. Early cancer was defined as those wherein cancer invasion reaches the submucosa, regardless of venous invasion or lymphatic invasion. Borrmann classification was applied to advanced cancer. All the colorectal tumor surgery and EMR specimens underwent clinical diagnosis in accordance with WHO classification, at the Pathological Department of the Akita Red Cross Hospital.

### Example 4

### Cytological Diagnosis

Regarding cytological diagnosis of colonic-mucous-layer detachment fluid, the colonic-mucous-layer detachment fluids collected at the Digestive Organ Center of the Akita Red Cross Hospital were stored in ThinPrep (registered trademark) PreservCyt Solution Vials (20 ml, prefilled/Box of 50 vials, order No. 0234005, CYTYC Corporation), formalin-fixated after centrifugation, and hematoxylin-eosin stained; then cytological diagnosis was conducted.

### Example 5

### Statistical Analysis

All statistical analysis and graph making were carried out by PRISM version 5 for Windows (Japanese version). With respect to methylation levels, an average value of the methylation level at each CpG region was determined to be the methylation level of that specimen, and examined in terms of clinical diagnosis and invasion depth. Regarding statistical significance, one-way analysis of variance was carried out in each group. As for correlation, t-test was conducted.

### Example 6

### ROC Curve

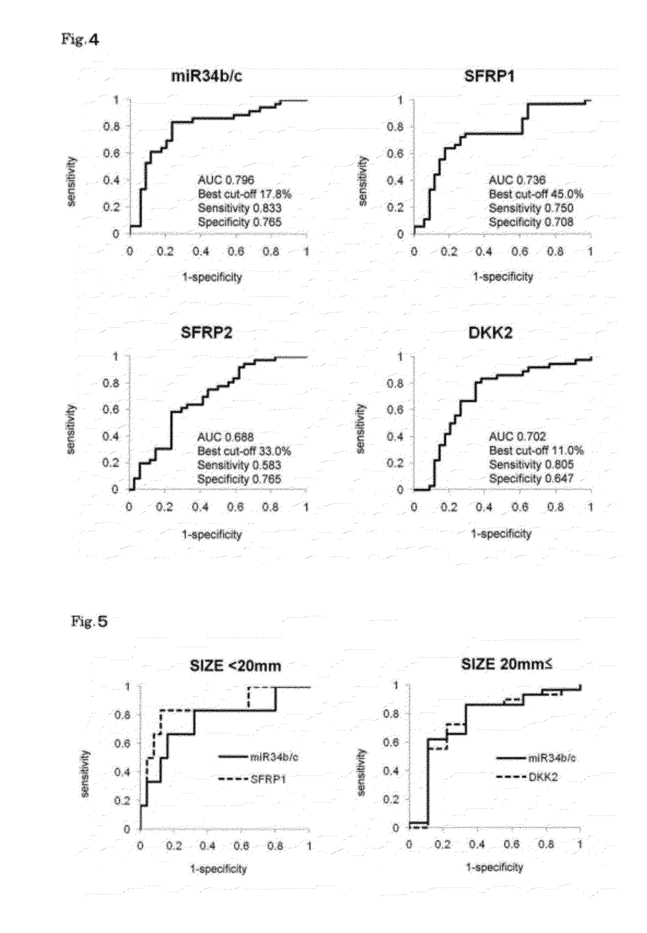

ROC curves were produced to determine threshold values of DNA methylation level of each gene mir34b/c, SFRP1, SFRP2 and DKK2 as an index for detecting invasive colorectal cancer (FIG. 4). Best cut-off values of mir34b/c, SFRP1, SFRP2 and DKK2 were 17.8%, 45%, 33% and 11%, respectively.

### Example 7

### ROC Curve for the Cases Wherein Tumor Diameter is Less than 20 mm or 20 mm or More

ROC curves were produced for the cases where tumor diameter is less than 20 mm, and for the cases where tumor diameter is 20 mm or more (FIG. 5). When the tumor diameter measured from the large intestinal lumen side is less than 20 mm, an ROC curve was produced using DNA methylation level of mir-34b/c gene and SFRP1 gene as indices (FIG. 5, left figure). In addition, when the tumor diameter measured from the large intestinal lumen side is 20 mm or more, an ROC curve was produced using DNA methylation level of mir-34b/c gene and DKK2 gene as indices (FIG. 5, right figure). These results show that differentiation of invasive tumor from non-invasive tumor when the tumor diameter is 20 mm or less is preferably carried out using DNA methylation level of mir34b/c gene or SFRP gene as the index, and that differentiation of invasive tumor from non-invasive tumor when the tumor diameter is 20 mm or more is preferably carried out using DNA methylation level of mir34b/c gene or DKK2 gene as the index.

### Example 8

### Diagnostic Flow Chart Wherein Tumor Size and DNA Methylation Level are Combined

A flow chart for diagnosing presence/absence of invasive tumors with the highest accuracy, by combining measurement of tumor diameter from the large intestinal lumen side and measurement of DNA methylation level, was made (FIG. 6). Using this flow chart, diagnosis of invasive tumor and non-invasive tumor can be made by starting from the leftmost box, wherein the judgment of tumor diameter of 20 mm or more (Yes) or that of less than 20 mm (No) is made, then by proceeding to the next box in accordance with the judgment criteria.

### Example 9

### Correlation of DNA Methylation Level in Colonic-Mucous-Layer Detachment Fluid and DNA Methylation Level in Biopsy Sample

Regarding both invasive tumor and non-invasive tumor, whether the percentage of methylated DNA of each gene mir34b/c, SFRP1, SFRP2 and DKK2 in colonic-mucous-layer detachment fluid correlates with that in biopsy sample was investigated. As a result, in invasive tumors, the percentage of methylated DNA of each gene mir34b/c, SFRP1, and SFRP2 in colonic-mucous-layer detachment fluid significantly correlated with that in biopsy sample with a risk rate of 3% or less. Furthermore, in all the tumors including invasive tumors and non-invasive tumors, the percentage of methylated DNA of each gene mir34b/c, SFRP1, SFRP2 and DKK2 in colonic-mucous-layer detachment fluid significantly correlated with that in biopsy sample with a risk rate of 5% or less.

### Example 10

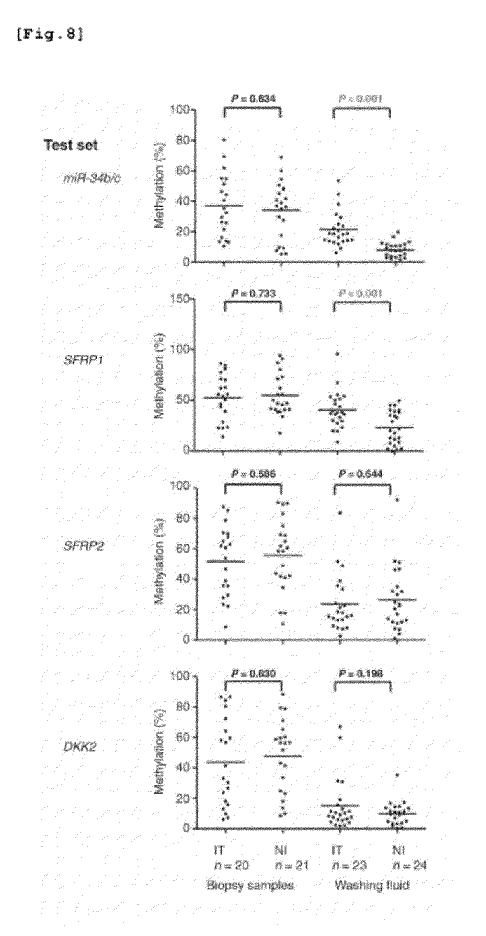

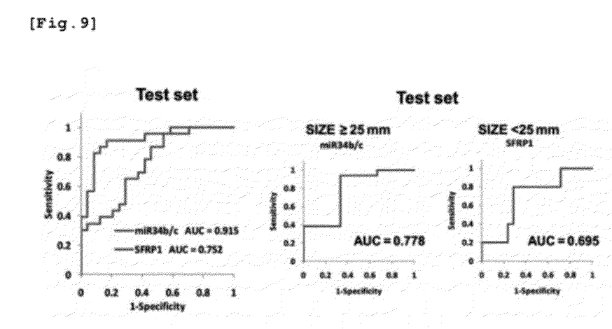

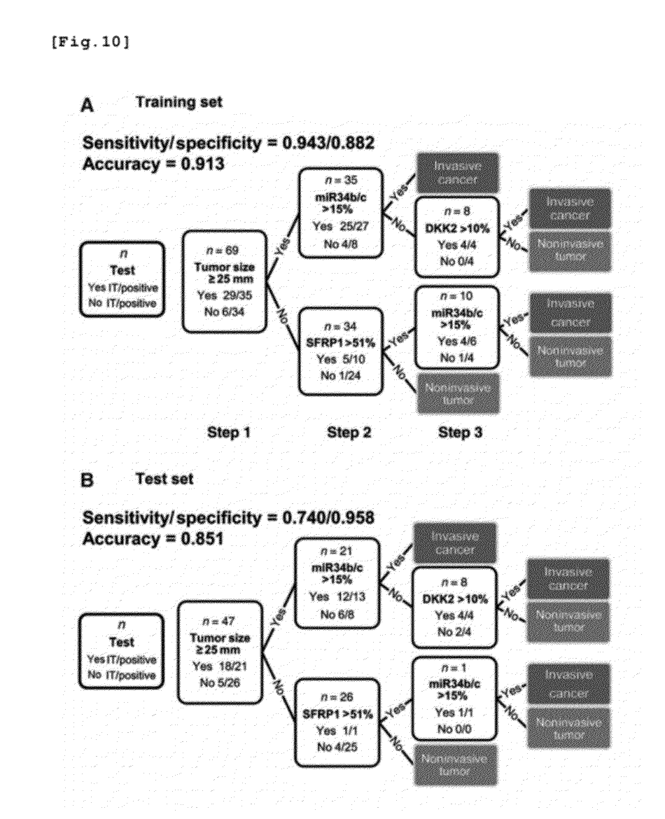

The same test as those described above was performed for the test set. Results are shown in FIGS. 8 and 9. Regarding statistically significant difference in the methylation level of each gene between invasive tumor and non-invasive tumor, results are as follows: for miR-34b/c gene, biopsy specimen exhibited P=0.634 and detachment fluid exhibited P<0.001; for SFRP1 gene, biopsy specimen exhibited P=0.733 and detachment fluid exhibited P=0.001; for SFRP2 gene, biopsy specimen exhibited P=0.586 and detachment fluid exhibited P=0.644; for DKK2 gene, biopsy specimen exhibited P=0.630 and detachment fluid exhibited P=0.198. Thus, except that no significant difference between invasive tumor and non-invasive tumor was observed for SFRP2, the results similar to the above-described results were obtained.

Regarding ROC analysis, miR-34b/c gene also exhibited very high AUC (0.915), with sensitivity/specificity of 0.870/0.875 at the cut off value of 13.0% and 0.565/0.958 at the cut off value of 17.8%. Regarding tumors with a tumor size of 25 mm or more, miR-34b/c showed AUC of 0.778; and for tumors with a tumor size of less than 25 mm, SFRP1 showed AUC of 0.695.

On the basis of these results, a diagnosis tree similar to that shown in FIG. 6 was made and shown in FIG. 10. In such a tree, at first groups are divided in terms of tumor size of 25 mm or more or less than 25 mm; then for those with 25 mm or more, groups are divided in terms of methylation level of miR-34b/c of more than 15% or 15% or less. When the methylation level is more than 15%, the tumor is determined as an invasive tumor, i.e., invasion depth is T1 or deeper. When the methylation level is 15% or less, then groups are divided in terms of methylation level of DKK2 of more than 10% or 10% or less, and when it is more than 10%, the tumor is determined to be an invasive tumor, and when it is 10% or less, then the tumor is determined to be a non-invasive tumor. Next, when tumor size is less than 25 mm, then groups are divided in terms of methylation level of SFRP1 of 51% or more or less than 51%, and when it is less than 51%, then the tumor is determined to be a non-invasive tumor; when it is more than 51%, then groups are divided in terms of methylation level of miR-34b/c of more than 15% or 15% or less; when it is more than 15%, the tumor is determined to be an invasive tumor, and when it is 15% or less, the tumor is determined to be a non-invasive tumor. With this diagnosis tree and using the training set data, the sensitivity/specificity was 0.943/0.882 and the accuracy was 0.913; using the test set data, the sensitivity/specificity was 0.740/0.958 and the accuracy was 0.851.

### INDUSTRIAL APPLICABILITY

The specimen, kit and method of the present invention is a revolutionary invention that can provide a method for non-invasively diagnosing invasiveness or degree of invasion of colorectal cancer or colorectal tumor by spraying a washing fluid onto the subject's colonic mucous layer to detach the mucus from the mucous layer, and collecting the detached mucous together with the washing fluid. By utilizing the specimen, kit and method of the invention, invasiveness or degree of invasion of colorectal cancer or colorectal tumor can be diagnosed non-invasively with high accuracy; thus they are useful for selecting a therapeutic method of colorectal cancer. Furthermore, by utilizing the specimen, kit and method of the invention, sensitivity to various drugs including anti-cancer agents can be investigated in advance. Moreover, by the specimen, kit and method of the invention, it becomes possible to evaluate therapeutic effects of a drug and/or a therapeutic method. By the specimen, kit and method of the invention, prediction of recurrence becomes possible. Accordingly, the specimen, kit and method of the invention are industrially applicable.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A colonic-mucous-layer detachment fluid comprising the colonic mucus detached from the colonic mucous layer, which does not substantially comprise a content existing in the digestive tract from the oral cavity to the small intestine and a component derived from said digestive tract.

35. The colonic-mucous-layer detachment fluid according to claim 34 for diagnosing invasive colorectal tumors, obtained by spraying a washing fluid directly onto the colonic mucous layer to detach the colonic mucus, and collecting the colonic mucus detached from the directly-sprayed site together with the washing fluid.

36. A method for collecting a colonic-mucous-layer detachment fluid for diagnosing invasive colorectal tumors, comprising:
(a) spraying a washing fluid directly onto the colonic mucous layer, and
(b) collecting the colonic mucus detached from the directly-sprayed site together with the washing fluid.

37. The method according to claim 36, wherein the washing fluid is a physiological isotonic solution.

38. The method according to claim 37, wherein the physiological isotonic solution is saline.

39. The method according to claim 36, wherein the spraying is carried out at a flow rate of 2-10 ml/s.

40. The method according to claim 36, wherein the washing fluid is sprayed without pre-washing.

41. A method for detecting an invasive colorectal tumor, comprising:
(a) determining one or more disease-related marker levels in the colonic-mucous-layer detachment fluid according to claim 34, and
(b) determining the presence or absence of an invasive colorectal tumor based on the disease-related marker levels.

42. The method according to claim 41, wherein the colonic mucous layer is a colonic mucous layer comprising a site of lesion.

43. The method according to claim 42, wherein the site of lesion is a site of lesion suspected of tumor invasion.

44. The method according to claim 41, wherein the determination of a disease-related marker level is carried out by cytological diagnosis.

45. The method according to claim 41, wherein the disease-related marker is methylated DNA.

46. The method according to claim 45, wherein the methylated DNA is a methylated DNA in the promoter region of one or more genes selected from the group consisting of SFRP1, SFRP2, DKK2, and hsa-mir-34b/c.

47. The method according to claim 41, wherein the disease-related marker is K-RAS mutation.

48. The method according to claim 41, comprising determining two or more disease-related marker levels.

49. A method for diagnosing the degree of invasion of an invasive colorectal tumor, comprising:
(a) determining one or more disease-related marker levels in the colonic-mucous-layer detachment fluid according to claim 34, and
(b) determining the degree of invasion based on the disease-related marker levels.

50. The method according to claim 49, wherein the disease-related marker level is a DNA methylation level.

51. A method for evaluating therapeutic effects of a drug and/or a therapeutic method, comprising:
(a) determining one or more disease-related marker levels A in the colonic-mucous-layer detachment fluid according to claim 34 before treatment by a drug and/or a therapeutic method,
(b) after the treatment by the drug and/or the therapeutic method, determining one or more disease-related marker levels B in the colonic-mucous-layer detachment fluid according to claims 34 which correspond to said A, and
(c) comparing said A with B.

52. A kit for collecting the colonic-mucous-layer detachment fluid according to claim 35, containing at least one sealable specimen collection container to collect the washing fluid, and a preservative solution.

53. The kit according to claim 52, further containing a tool for treating the collected colonic-mucous-layer detachment fluid.

54. The kit according to claim 53, wherein the tool for treating the colonic-mucous-layer detachment fluid is one or more primers used for the detection of methylated DNA.
